SEQUENCE LISTING (12 sequences)

SEQ ID NO: 1 - protein, 5 aa, Artificial Sequence
  HCDR1
NYGLS

SEQ ID NO: 2 - protein, 17 aa, Artificial Sequence
  HCDR2
WISANNGDTNYGQEFQG

SEQ ID NO: 3 - protein, 13 aa, Artificial Sequence
  HCDR3
DSSSSWARWFFDL

SEQ ID NO: 4 - protein, 11 aa, Artificial Sequence
  LCDR1
GGNIIGSKLVH

SEQ ID NO: 5 - protein, 7 aa, Artificial Sequence
  LCDR2
DDGDRPS

SEQ ID NO: 6 - protein, 11 aa, Artificial Sequence
  LCDR3
QVWDTGSDPVV

SEQ ID NO: 7 - DNA, 366 bp, Artificial Sequence
  cDNA heavy chain variable domain
caggtccagctggtgcagtctggggctgaggtgaagaagcctggggcctcagtgaaggtctcctgcaaggcttctggttacacctttacaaattatggtctcagctgggtgcgacaggcccctggacaagggcttgagtggatgggatggatcagcgctaataatggcgacacaaattatggacaggaattccagggcagagtcaccatgaccacagatacatccacgagcacagcctacatggagttgaggagcctgagatctgacgacacggccgtttattactgtgcgagagactccagcagcagctgggcccgctggtttttcgatctctggggccgggggacactggtcaccgtctcctca

SEQ ID NO: 8 - protein, 122 aa, Artificial Sequence
  Polypeptide sequence heavy chain variable region
QVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSS

SEQ ID NO: 9 - DNA, 327 bp, Artificial Sequence
  cDNA light chain variable domain
tcctatgtgctgactcagccaccctcggtgtcagtggccccaggaaagacggccaggattacctgtgggggaaacatcattggaagtaaacttgtacactggtaccagcagaagccaggccaggcccctgtgctggtcatctatgatgatggcgaccggccctcagggatccctgagcgattctctggctccaactctgggaacacggccaccctgaccatcagcagggtcgaggccggggatgaggccgactattattgtcaggtgtgggatactggtagtgatcccgtggtattcggcggagggaccaagctgaccgtcctaggt

SEQ ID NO: 10 - protein, 108 aa, Artificial Sequence
  Polypeptide sequence light chain variable region
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVL

SEQ ID NO: 11 - protein, 449 aa, Artificial Sequence
  Heavy chain
QVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 214 aa, Artificial Sequence
  Light chain
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2025/0059269 A1
(43) Pub. Date: Feb. 20, 2025

(54) METHODS FOR TREATING ATOPIC DERMATITIS AND RELATED DISORDERS

(71) Applicant: Medimmune Limited, Cambridge, Cambridgeshire (GB)

(72) Inventors: Thomas MARK, Ballerup (DK); Lisa SOLDBRO, Ballerup (DK); Cheistian Bjerregaard ØLAND, Ballerup (DK)

(21) Appl. No.: 18/566,712

(22) PCT Filed: Jun. 10, 2022
(86) PCT No.: PCT/EP2022/065851

§ 371 Date: Dec. 4, 2023

(30) Foreign Application Priority Data
Jun. 11, 2021 (GB) 2108379.5

(51) Int. Cl. C07K 16/24

(52) U.S. Cl. CPC C07K 16/244; C07K 2317/24

(57) ABSTRACT

The present invention relates to methods for treating atopic dermatitis in a subject using an interleukin-13 (IL-13) binding protein, such as an anti-IL-13 antibody or an IL-13-binding fragment thereof.

### CROSS REFERENCE TO RELATED APPLICATIONS

This application is a national stage application of PCT/EP2022/065851, filed Jun. 10, 2022, which claims priority to GB Application No. 2108379.5, filed Jun. 11, 2021, each of which are hereby incorporated by reference in their entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 10,576 Byte ASCII (Text) filed named “42639-251-ST25,” created on Dec. 1, 2023.

### FIELD OF THE INVENTION

The present invention relates to methods for treating atopic dermatitis in a subject using an interleukin-13 (IL-13) binding protein, such as an anti-IL-13 antibody or an IL-13-binding fragment thereof.

### BACKGROUND TO THE INVENTION

Atopic dermatitis (AD) is a heterogeneous inflammatory skin disease arising from genetic and environmental factors that disrupt skin barrier function and immune response (Leung, D. Y. and Guttman-Yassky, E. Deciphering the complexities of atopic dermatitis: shifting paradigms in treatment approaches. J Allergy Clin Immunol. 2014; 134:769-779). Current management generally involves treatment combinations to suppress inflammation, restore skin barrier function, and prevent superinfection (Wollenberg, A., Oranje, A., Deleuran, M., Simon, D., Szalai, Z., Kunz, B. et al. ETFAD/EADV Eczema task force 2015 position paper on diagnosis and treatment of atopic dermatitis in adult and paediatric patients. J Eur Acad Dermatol Venereol. 2016; 30:729-747).

Topical corticosteroids (TCSs) are overwhelmingly the most frequently prescribed class of drugs for AD patients, although long-term application of a TCS is not recommended. Topical calcineurin inhibitors (TCI) are generally effective and safe as short-term treatments. Skin malignancies and increased risk of lymphomas have prompted regulatory authorities to require a warning regarding the long-term safety of topical tacrolimus and pimecrolimus in their prescribing information, for example. First generation antihistamines are widely prescribed for acute symptomatic treatment of pruritus (itching), although their effectiveness is limited and largely attributed to their sedating effect. Oral immunosuppressants and glucocorticoids are effective, but are sometimes associated with severe toxicity and side effects, thus limiting their use to short term and/or intermittent therapy.

Cyclosporine A (CSA), a therapy for severe AD in some territories, is an immunosuppressant affecting both humoral and cellular immune responses, which increases susceptibility to infections and decreases cancer immunosurveillance. Other commonly recognized toxicities include hypertension and impaired renal and hepatic function. In addition, CSA interacts with other commonly used drugs, potentially affecting their metabolism and effect. Systemic immunosuppressants are typically reserved for treatment of moderate-to-severe AD because of their associated with adverse events and unsuitability for long-term use (Wollenberg, A., Oranje, A., Deleuran, M., Simon, D., Szalai, Z., Kunz, B. et al. ETFAD/EADV Eczema task force 2015 position paper on diagnosis and treatment of atopic dermatitis in adult and paediatric patients. J Eur Acad Dermatol Venereol. 2016; 30:729-747). Therefore more effective and well-tolerated therapies are required that target the mechanisms of AD pathophysiology rather than simply providing symptom relief.

A key feature of AD is upregulation of IL-13 and interleukin-4 (IL-4) in lesional and nonlesional skin, suggesting both cytokines can contribute to AD pathogenesis (see Nomura, I., Goleva, E., Howell, M. D., Hamid, Q. A., Ong, P. Y., Hall, C. F. et al. Cytokine milieu of atopic dermatitis, as compared to psoriasis, skin prevents induction of innate immune response genes. J Immunol. 2003; 171:3262-3269; Tazawa, T., Sugiura, H., Sugiura, Y., and Uehara, M. Relative importance of IL-4 and IL-13 in lesional skin of atopic dermatitis. Arch Dermatol Res. 2004; 295:459-464). Moreover, AD severity is associated with increased IL-13 and associated chemokine mRNA and serum levels, whereas reductions in IL-13 concentrations have correlated with treatment response and improved clinical outcomes. Although treatment with dupilumab, a human mAb that inhibits both IL-4 and IL-13 signaling, has demonstrated improvements in AD symptoms, the relative contribution of each of these cytokines to AD pathogenesis is unclear.

In real-world clinical practice, the prevalence of conjunctivitis in patients receiving dupilumab has been estimated to be 26% based on a systematic review of the literature (Halling A S et al. Real-world evidence of dupilumab efficacy and risk of adverse events: A systematic review and meta-analysis. J Am Acad Dermatol 2020)) and can be persistent despite adequate ophthalmological treatment (Achten R et al. Long-term follow-up and treatment outcomes of conjunctivitis during dupilumab treatment in patients with moderate-to-severe atopic dermatitis. J Allergy Clin Immunol Pract 2020). Additionally, the incidence of ocular complications with dupilumab, such as conjunctivitis, has been shown to increase with AD severity (Simpson EL et al. Two Phase 3 Trials of Dupilumab versus Placebo in Atopic Dermatitis. N Engl J Med 2017; 376:1090-1091; Thyssen J P et al. Incidence, prevalence, and risk of selected ocular disease in adults with atopic dermatitis. J Am Acad Dermatol 2017; 77:280-286 e281). Ophthalmological side effects during dupilumab treatment have only been observed in patients with AD and not in studies of chronic sinusitis with nasal polyps, asthma or eosinophilic esophagitis (Bachert C et al. Effect of subcutaneous dupilumab on nasal polyp burden in patients with chronic sinusitis and nasal polyposis: a randomized clinical trial. JAMA 2016; 315:469-479; Castro M et al. Dupilumab Efficacy and Safety in Moderate-to-Severe Uncontrolled Asthma. N Engl J Med 2018; 378:2486-2496; Hirano I et al. Efficacy of dupilumab in a phase 2 randomized trial of adults with active eosinophilic esophagitis. Gastroenterology 2020; 158:111-122.e110), suggesting that there are AD-specific predisposing factors. Since the introduction of dupilumab as a treatment for AD, conjunctivitis has been identified as an important adverse event, which has resulted in an increased collaboration with ophthalmologists (Agnihotri G et al. A Clinician's Guide to the Recognition and Management of Dupilumab-Associated Conjunctivitis. Drugs in R&D 2019; 19:311-318; Wollenberg et al. 2018; Wollenberg A et al. Conjunctivitis occurring in atopic dermatitis patients treated with dupilumab-clinical characteristics and treatment. J Allergy Clin Immunol Pract 2018).

IL-13 is a 114 amino acid cytokine with an unmodified molecular mass of approximately 12 kDa. IL-13 is most closely related to IL-4 with which it shares 30% sequence homology at the amino acid level. The human IL-13 gene is located on chromosome 5q31 adjacent to the IL-4 gene. Although initially identified as a Th2 CD4+lymphocyte derived cytokine, IL-13 is also produced by Th1 CD4+T-cells, CD8+T lymphocytes NK cells, and non-T-cell populations such as mast cells, basophils, eosinophils, macrophages, monocytes and airway smooth muscle cells. IL-13 has been linked with a number of diseases, in particular, diseases which are caused by an inflammatory response. For example, administration of recombinant IL-13 to the airways of naive non-sensitised rodents was shown to cause many aspects of the asthma phenotype including airway inflammation, mucus production and airways hyper-responsiveness. A similar phenotype was observed in a transgenic mouse in which IL-13 was specifically overexpressed in the lung. In this model, more chronic exposure to IL-13 also resulted in fibrosis.

A number of genetic polymorphisms in the IL-13 gene have also been linked to allergic diseases. In particular, a variant of the IL-13 gene in which the arginine residue at amino acid 130 is substituted with glutamine (Q130R) has been associated with bronchial asthma, atopic dermatitis and raised serum IgE levels. This particular IL-13 variant is also referred to as the Q110R variant (arginine residue at amino acid 110 is substituted with glutamine) by some groups who exclude the 20 amino acid signal sequence from the amino acid count.

Tralokinumab (also known as CAT-354 and BAK502G9) is a fully human therapeutic antibody that binds to and neutralizes IL-13, including the Q130R variant (see Popovic et al. J. Mol. Biol. (2017) 429(2):208-219; May, R. D., Monk, P. D., Cohen, E. S., Manuel, D., Dempsey, F., Davis, N. H. et al. Preclinical development of CAT-354, an IL-13 neutralizing antibody, for the treatment of severe uncontrolled asthma. Br J Pharmacol. 2012; 166:177-193).

Tralokinumab has previously been tested in phase 2b study of 204 adults for the treatment of AD—where patients received 45 mg, 150 mg, or 300 mg of subcutaneous tralokinumab, or placebo, every 2 weeks for 12 weeks with concomitant topical glucocorticoids—and was found to improve change from baseline in Eczema Area Severity Index (EASI) score, together with improvements in Scoring atopic dermatitis (SCORAD), Dermatology Life Quality Index (DLQI), and pruritus numeric rating scale scores, as compared to placebo (Wollenberg J. Allergy Clin. Immunol. (2019) 143(1):135-141).

There remains a desire in the art for further and improved treatments for AD that address, for example, at least some of the concerns referred to above.

### SUMMARY OF THE INVENTION

The inventors have found that patients in which AD is inadequately controlled by therapeutic agents targeting both IL-13 and IL-4 signaling (e.g. an antibody like dupilumab) can be effectively treated with IL-13 binding proteins (e.g. an anti-IL13 antibody like tralokinumab).

Thus, in one aspect, the invention provides an interleukin-13 (IL-13) binding protein for use in a method of treating atopic dermatitis (AD) in a subject, wherein the AD is inadequately controlled by an agent that inhibits IL-13 and IL-4 signalling and wherein the method comprises administering the IL-13 binding protein to the subject.

In some embodiments, the method comprises selecting a subject whose AD is inadequately controlled by an agent that inhibits IL-13 and IL-4 signalling.

In another aspect, the invention provides a method of treating atopic dermatitis (AD) in a subject in need thereof, wherein the AD is inadequately controlled by an agent that inhibits IL-13 and IL-4 signalling, and wherein the method comprises the step of administering the IL-13 binding protein to the subject.

In some embodiments, the method comprises selecting a subject whose AD is inadequately controlled by an agent that inhibits IL-13 and IL-4 signalling.

In another aspect, the invention provides use of an IL-13 binding protein in the manufacture of a medicament for treating AD in a subject, wherein the AD is inadequately controlled by an agent that inhibits IL-13 and IL-4 signaling.

In any of the aspects or embodiments described herein, the AD may be moderate-to-severe AD or severe AD.

In some aspects, the invention provides an IL-13 binding protein for use in a method of treating atopic dermatitis in a subject who has experienced conjunctivitis when treated with an agent that inhibits IL-13 and IL-4 signalling, wherein the method comprises administering the IL-13 binding protein to the subject.

In some aspects, the invention provides a method for treating atopic dermatitis in a subject who has experienced conjunctivitis when treated with an agent that inhibits IL-13 and IL-4 signaling, wherein the method comprises administering the IL-13 binding protein to the subject.

In some aspects, the invention provides the use of an IL-13 binding protein in the manufacture of a medicament for treating atopic dermatitis in a subject who has experienced conjunctivitis when treated with an agent that inhibits IL-13 and IL-4 signaling, wherein the method comprises administering the IL-13 binding protein to the subject.

In some embodiments, the method comprises selecting a subject who has experienced conjunctivitis when treated with an agent that inhibits IL-13 and IL-4 signaling.

In any of the aspects or embodiments described herein, the AD may be inadequately controlled by cyclosporine A or the subject may have contraindications to cyclosporine A.

In any of the aspects or embodiments described herein, the method may comprise steps of: (a) administering a first dose of the IL-13 binding protein to the subject; and (b) administering one or more secondary dose(s) of the IL-13 binding protein to the subject, wherein each secondary dose is administered to the subject from 12 days to 35 days after the immediately preceding dose. In preferred embodiments, each secondary dose is administered to the subject about 12-16 days after the immediately preceding dose, or from 25 days to 31 days after the immediately preceding dose. In particularly preferred embodiments, each secondary dose is administered to the subject about 2 weeks after the immediately preceding dose, or about 4 weeks after the immediately preceding dose.

In some embodiments, the methods described herein are carried out for at least 2 weeks, at least 3 weeks, at least 12 weeks, at least 3 months, at least 16 weeks, at least 24 weeks, at least 26 weeks, at least 6 months, at least 32 weeks, at least 36 weeks, at least a year, or at least 52 weeks or more. In preferred embodiments, the methods are carried out for at least 26 weeks.

In any of the aspects or embodiments described herein, the method may comprise the steps of: (a) administering a first dose of about 10 to about 600 mg of the IL 13 binding protein to the subject; and (b) administering one or more secondary dose(s) of about 10 to about 600 mg of the IL-13 binding protein to the subject, wherein each secondary dose is administered to the subject from 12 days to 35 days after the immediately preceding dose.

In any of the aspects or embodiments described herein, the method may comprise the steps of: (a) administering a first dose of about 600 mg of the IL 13 binding protein to the subject; and (b) administering one or more secondary dose(s) of about 300 mg of the IL-13 binding protein to the subject, wherein each secondary dose is administered to the subject from 12 days to 35 days after the immediately preceding dose.

In a preferred embodiment, each secondary dose is administered to the subject about 12-16 days after the immediately preceding dose, or from 25 days to 31 days after the immediately preceding dose. In a further preferred embodiment, each secondary dose is administered to the subject about 2 weeks after the immediately preceding dose, or about 4 weeks after the immediately preceding dose.

In preferred embodiments, the IL-13 binding protein is an anti-IL-13 antibody, or an IL-13-binding fragment thereof.

In some embodiments, the IL-13 binding protein for use according to any of the methods described herein is an anti-IL-13 antibody, or IL-13-binding fragment thereof, wherein the anti-IL-13 antibody, or IL-13-binding fragment thereof comprises:
a heavy chain complementarity determining region 1 (HCDR1) comprising an amino acid sequence of SEQ ID NO:1;
a heavy chain complementarity determining region 2 (HCDR2) comprising an amino acid sequence of SEQ ID NO:2;
a heavy chain complementarity determining region 3 (HCDR3) comprising an amino acid sequence of SEQ ID NO:3;
a light chain complementarity determining region 1 (LCDR1) comprising an amino acid sequence of SEQ ID NO:4;
a light chain complementarity determining region 2 (LCDR2) comprising an amino acid sequence of SEQ ID NO:5; and
a light chain complementarity determining region 3 (LCDR3) comprising an amino acid sequence of SEQ ID NO:6.

In further embodiments, the anti-IL-13 antibody, or IL-13-binding fragment thereof, comprises a heavy chain variable region (HCVR) and a light chain variable region (LCVR), wherein:
(i) the heavy chain variable region comprises:
a heavy chain complementarity determining region 1 (HCDR1) comprising an amino acid sequence of SEQ ID NO:1;
a heavy chain complementarity determining region 2 (HCDR2) comprising an amino acid sequence of SEQ ID NO:2; and
a heavy chain complementarity determining region 3 (HCDR3) comprising an amino acid sequence of SEQ ID NO:3; and
(ii) the light chain variable region comprises:
a light chain complementarity determining region 1 (LCDR1) comprising an amino acid sequence of SEQ ID NO:4;
a light chain complementarity determining region 2 (LCDR2) comprising an amino acid sequence of SEQ ID NO:5; and
a light chain complementarity determining region 3 (LCDR3) comprising an amino acid sequence of SEQ ID NO:6.

In some embodiments, the anti-IL-13 antibody, or IL-13-binding fragment thereof comprises a heavy chain variable region sequence of SEQ ID NO:8 and a light chain variable region sequence of SEQ ID NO:10.

In some embodiments, the anti-IL-13 antibody, or IL-13-binding fragment thereof comprises a heavy chain of SEQ ID NO:11 and a light chain sequence of SEQ ID NO:12.

In a preferred embodiment, the anti-IL-13 antibody is tralokinumab.

The IL-13 binding protein may be administered as a monotherapy or as a combination therapy with a second therapeutic agent. In preferred embodiments, the IL-13 binding protein is administered in combination with a topical corticosteroid.

The subject treated by the IL-13 binding protein in the methods described herein have AD, wherein the AD is inadequately controlled by an agent that inhibits IL-13 and IL-4 signalling. In preferred embodiments, the agent that inhibits IL-13 and IL-4 signalling may be an antibody or antigen binding fragment thereof. In more preferred embodiments, the agent that inhibits IL-13 and IL-4 signalling is an antibody or antigen binding fragment thereof that binds to IL-4Rα. In even more preferred embodiments, the agent that inhibits IL-13 and IL-4 signalling is dupilumab.

It is a goal of the methods of described herein to achieve ≥50% improvement of Eczema Area and Severity Index (EASI-50) compared to baseline. In preferred embodiments, the methods described herein achieve ≥75% improvement of Eczema Area and Severity Index (EASI-75) compared to baseline. In some embodiments, EASI-50 or EASI-75 is achieved at week 16. In some embodiments, EASI-50 or EASI-75 is achieved at week 26.

For example, in one embodiment, the method comprises (a) administering a first dose of about 600 mg of the IL-13 binding protein to the subject; and (b) administering one or more secondary dose(s) of about 300 mg of the IL-13 binding protein to the subject, wherein each secondary dose is administered to the subject 2 weeks after the immediately preceding dose, wherein the IL-13 binding protein is an antibody comprising a heavy chain variable region sequence of SEQ ID NO:8 and a light chain variable region sequence of SEQ ID NO:10 (e.g. tralokinumab), wherein the method is carried out for at least 26 weeks and wherein the method achieves ≥75% improvement of Eczema Area and Severity Index (EASI-75) compared to baseline at 26 weeks. In some embodiments, (i) the IL-13 binding protein is administered in combination with a topical corticosteroid and/or (ii) the AD is inadequately controlled by cyclosporine A or the subject has contraindications to cyclosporine A.

### DESCRIPTION OF FIGURES

FIG. 1 shows the trial design for the ECZTRA7 trial.

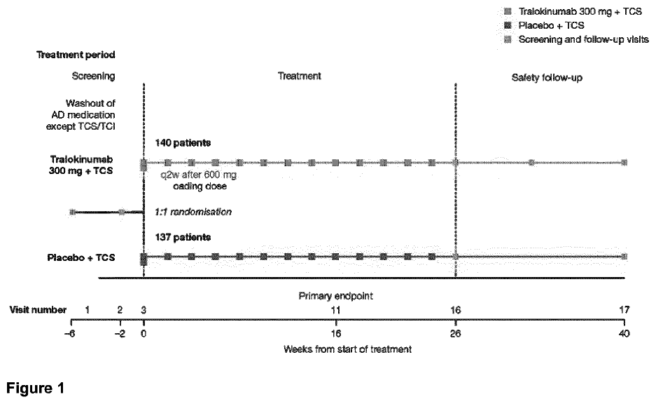

### DETAILED DESCRIPTION

The invention relates to methods for treating atopic dermatitis (AD) in a subject using an interleukin-13 (IL-13) binding protein (e.g. an anti-IL-13 antibody or an IL-13-binding fragment thereof, such as tralokinumab), wherein the AD is inadequately controlled by an agent which inhibits IL-13 and IL-4 signaling (such as dupilumab). Without being bound by any particular theory, the inventors have found that AD can be effectively treated in a subject by administration of an IL-13 binding protein, even when the AD is not adequately controlled by agents that inhibit both IL-13 and IL-4 signaling.

### Atopic Dermatitis

“Atopic dermatitis” (AD), as used herein, means an inflammatory skin disease characterized by intense pruritus (e.g. severe itch) and by scaly and dry eczematous lesions.

The term “atopic dermatitis” includes AD caused by or associated with epidermal barrier dysfunction, allergy (e.g. allergy to certain foods, pollen, maid, dust mite, animals, etc.), radiation exposure, and/or asthma. In some embodiments, the present invention relates to moderate-to-severe or severe AD.

As used herein, “moderate-to-severe AD” is characterized by intensely pruritic, widespread skin lesions that are often complicated by persistent bacterial, viral or fungal infections.

Moderate-to-severe AD also includes chronic AD. In many cases, the chronic lesions include thickened plaques of skin, lichenification and fibrous papules. In general, patients affected by moderate-to-severe AD also have more than 20% of the body's skin affected, or 10% of skin area in addition to involvement of the eyes, hands and body folds. Moderate-to-severe AD is also considered to be present in patients who frequently require treatment with a topical corticosteroid. In the clinical studies reported herein a subject with “moderate to severe AD” was a subject having an IGA score of 3-4.

As used herein, “severe AD” refers to chronic relapsing AD that is refractory to treatment with medium-potency and high-potency TCS and/or immunosuppressant therapy. Severe AD is also characterized by chronic intensely pruritic lesions affecting more than 20% of the body surface area. Severe AD can be considered to be present in subjects with chronic AD according to the Eichenfield criteria (Eichenfield et al 2014, J. Am. Acad. Dermatol. 70:338-351), for which treatment with a potent topical corticosteroid (TCS) is indicated, and/or where the subject is resistant to treatment with a systemic corticosteroid and/or non-steroidal immunosuppressant. In the clinical studies reported herein a subject with “severe AD” was a subject having an IGA score of 4. Thus, in certain embodiments, the method treats severe AD in a subject, where the subject has an IGA score of 4 at baseline. A subject with “severe AD” may have AD on at least 10% of their body surface area at screening and baseline, an EASI score of ≥20 at screening and baseline, and an IGA score of ≥3 at screening and baseline.

### Subject

As used herein, the term “subject” includes human and non-human animals, particularly mammals. Typically, the subject is a human, as shown in the examples below.

Subjects treated by the methods of this invention are those with AD (especially moderate-to-severe AD or severe AD) who have previously been treated with a therapeutic agent that inhibits both IL-13 and IL-4 signalling, such as an anti-IL4Rα antibody (e.g. dupilumab), but their AD is inadequately controlled with this agent.

In some embodiments, “inadequately controlled” by an agent that inhibits IL-13 and IL-4 signalling means that the AD is non-responsive, resistant or refractory to an agent that inhibits IL-13 and IL-4 signalling”, such as an anti-IL4Rα antibody (e.g. dupilumab). In such embodiments, treatment of a subject with AD with an agent that inhibits IL-13 and IL-4 signalling, such as an anti-IL4Rα antibody (e.g. dupilumab) did not have a therapeutic effect. For example, a subject with moderate-to-severe AD or severe AD (such as those with chronic relapsing AD) that has been treated with an agent that inhibits IL-13 and IL-4 signalling (e.g. dupilumab) did not show a decrease in one or more AD-associated parameter score(s), as described in more detail below. The time for the assessment of a therapeutic effect will vary depending on the typical timeframe for onset of action of the agent that inhibits IL-13 and IL-4.

In some embodiments, the term “inadequately controlled”, “non-responsive”, “resistant” or “refractory” to an agent that inhibits IL-13 and IL-4 signalling (e.g. dupilumab) refers to a subject with AD that has been treated with an agent that inhibits IL-13 and IL-4 signalling (e.g. dupilumab), but wherein the agent did not have a therapeutic effect e.g. wherein the method did not achieve ≥50% improvement of Eczema Area and Severity Index (EASI-50) compared to baseline or >75% improvement of Eczema Area and Severity Index (EASI-75) compared to baseline, for example at week 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52.

In some embodiments, the agent that inhibits IL-13 and IL-14 signalling is an antibody. In preferred embodiments, the agent that inhibits IL-13 and IL-14 signalling is dupilumab.

In some embodiments, dupilumab treatment has been deemed not medically advisable by a physician for the subject. Such a subject may be identified by the following criteria: (1) not currently a candidate for dupilumab treatment due to: medical contraindication(s); hypersensitivity to dupilumab or excipient(s); use of concomitant medications prohibited with dupilumab; (2) previous intolerance, side effects and/or unacceptable toxicity on previous exposure to dupilumab; and/or (3) requirement for dupilumab at doses or duration beyond that specified in the prescribing information.

Common side effects associated with dupilumab include conjunctivitis, eosinophilia; eye inflammation; eye pruritus; headache; oral herpes. In some embodiments, use of dupilumab has been discontinued in a subject due to adverse side effects, for example due to conjunctivitis.

In some aspects, the invention provides an IL-13 binding protein for use in a method of treating atopic dermatitis in a subject who has experienced conjunctivitis when treated with an anti-IL4Rα antibody or an antibody that inhibits IL-4 and IL-13 signalling, e.g. dupilumab, wherein the method comprises administering the IL-13 binding protein to the subject.

In some aspects, the invention provides an IL-13 binding protein for use in a method of treating atopic dermatitis in a subject, wherein the method comprises the steps of: (a) selecting a subject who has experienced conjunctivitis when treated with an anti-IL4Rα antibody or an antibody that inhibits IL-4 and IL-13 signalling, e.g. dupilumab; and (b) administering the IL-13 binding protein to the subject.

In some aspects, the invention provides a method for treating atopic dermatitis in a subject who has experienced conjunctivitis when treated with an anti-IL4Rα antibody or an antibody that inhibits IL-4 and IL-13 signalling, e.g. dupilumab, wherein the method comprises administering the IL-13 binding protein to the subject.

In some aspects, the invention provides a method for treating atopic dermatitis in a subject, wherein the method comprises the steps of: (a) selecting a subject who has experienced conjunctivitis when treated with an anti-IL4Rα antibody or an antibody that inhibits IL-4 and IL-13 signalling, e.g. dupilumab; and (b) administering the IL-13 binding protein to the subject.

In some aspects, the invention provides the use of an IL-13 binding protein in the manufacture of a medicament for treating atopic dermatitis in a subject who has experienced conjunctivitis when treated with an anti-IL4Rα antibody or an antibody that inhibits IL-4 and IL-13 signalling, e.g. dupilumab, wherein the method comprises administering the IL-13 binding protein to the subject.

In some aspects, the invention provides the use of an IL-13 binding protein in the manufacture of a medicament for treating atopic dermatitis in a subject, wherein the method comprises the steps of: (a) selecting a subject who has experienced conjunctivitis when treated with an anti-IL4Rα antibody or an antibody that inhibits IL-4 and IL-13 signalling, e.g. dupilumab; and (b) administering the IL-13 binding protein to the subject.

A subject with AD (especially moderate-to-severe AD or severe AD) may also be resistant, non-responsive or inadequately responsive to treatment with a non-steroid systemic immunosuppressant. The term “non-steroid systemic immunosuppressant” includes cyclosporine A (CSA), methotrexate, mycophenolate mofetil, azathioprine, and interferon-gamma. In certain embodiments, the term also includes immunobiologics such as tumor necrosis factor alpha (TNFa) inhibitors (e.g. an anti-TNFa antibody such as infliximab), CD11a inhibitors (e.g. an anti-CD11a antibody such as efalizumab), IgE inhibitors (e.g. omalizumab), CD20 inhibitors (e.g. rituximab). Thus, in some cases, the methods described herein may treat AD in subjects that are resistant, nonresponsive (refractory) or inadequately responsive to treatment with a systemic immunosuppressant. The term “resistant, non-responsive or inadequately responsive to a systemic immunosuppressant” refers to a subject with AD that has been treated with a systemic immunosuppressant and the immunosuppressant did not have a therapeutic effect, e.g. a subject with moderate-to-severe AD or severe AD (such as those with chronic relapsing AD) that has been treated with a non-steroid systemic immunosuppressant for between 1-3 months and did not show a decrease in one or more AD-associated parameter score(s). The time for the assessment of a therapeutic effect will vary depending on the typical timeframe for onset of action of the non-steroid systemic immunosuppressant. Such timeframes are well known. For example, for cyclosporine the onset of action is typically 2-6 weeks, but for other non-steroid systemic immunosuppressants it is typically around 8-12 weeks.

In some embodiments, immunosuppressant treatment has been deemed not medically advisable by a physician for the subject. Such a subject may be identified by the following criteria: (1) not currently a candidate for immunosuppressant treatment due to: medical contraindication(s); or hypersensitivity to the immunosuppressant or excipient(s); use of concomitant medications prohibited with immunosuppressant; or increased susceptibility to immunosuppressant induced renal damage or increased risk of serious infections; (2) previous intolerance and/or unacceptable toxicity on previous exposure to an immunosuppressant; and/or (3) requirement for immunosuppressant at doses or duration beyond that specified in the prescribing information.

In any of the methods described herein for the treatment of atopic dermatitis, e.g. severe atopic dermatitis, the subject may be one in which the atopic dermatitis is not adequately controlled by cyclosporine A (CSA), e.g. oral cyclosporine A, or the subject has contraindications to cyclosporine A (CSA), e.g. oral cyclosporine A.

An inadequate response to CSA is defined as flare of AD on CSA tapering after a maximum of 6 weeks of high dose (5 mg/kg/day) to maintenance dose (2 to 3 mg/kg/day) or a flare after a minimum of 3 months on maintenance dose. Flare is defined as increase in signs or symptoms leading to escalation of therapy, which could be an increase in dose, a switch to a higher potency class of TCS, or the start of another systemic non-steroidal immunosuppressive drug.

Contraindications to CSA include:
i. medical contraindications (e.g. uncontrolled hypertension on medication) or hypersensitivity to CSA active substance or excipients;
ii. use of prohibited concomitant medications (e.g. statins, digoxin, macrolide, antibiotics, barbiturates, anti-seizure, non-steroidal anti-inflammatory drugs, diuretics, angiotensin-converting-enzyme inhibitors, St John's Wort);
iii. increased susceptibility to CSA-induced renal damage (elevated creatinine) and liver damage (elevated function tests), or increased risk of serious infections;
iv. intolerance or unacceptable toxicity (e.g. elevated creatinine, elevated liver function tests, uncontrolled hypertension, paraesthesia, headache, nausea, hypertrichosis), or
v. requirement for CSA at doses >5 mg/kg/day, or duration beyond those specified in the prescribing information (>1 year).

### IL-13 and IL-4 Signalling Inhibiting Agents

The invention relates to IL-13 binding proteins for treating AD in subjects, wherein the AD is not adequately controlled by an agent that inhibits IL-13 and IL-4 signalling.

Agents that inhibit IL-13 and IL-4 signalling include for example, antibodies or antigen binding fragments thereof, as well as small molecule inhibitors and IL-4 muteins.

Preferably, in one embodiment, the agent that inhibits IL-4 and IL-13 signalling is an antibody or antigen binding fragment thereof. The antigen binding fragment can be selected from a Fab, Fab′, F(ab′)2, Fd, Fv, single-chain Fv (scFv), or disulfide-linked Fvs (sdFv).

In some embodiments, the agent that inhibits IL-13 and IL-4 signalling is an antibody or antigen binding fragment thereof that binds to IL-4Rα. Examples of antibodies that bind to IL-4Rα include dupilumab. In preferred embodiments, the antibody that inhibits IL-13 and IL-4 signalling is dupilumab.

### Treatment of AD

The methods described herein treat AD. Generally, the terms “treat”, “treating”, “treatment”, or the like, mean to alleviate (reduce, minimise, or eliminate) symptoms, or to reduce, minimise or eliminate the causation of symptoms either on a temporary or permanent basis.

### AD-Associated Parameters

Various AD-associated parameters are available to measure the severity of AD and the impact of a drug on AD. These include Investigators Global Assessment (IGA); Eczema Area and Severity Index (EASI); SCORing Atopic Dermatitis (SCORAD); and/or pruritus Numeric Rating Scale (NRS). The methods described herein may improve in an AD-associated parameter in the subject. Alternately, the methods may maintain improvement in an AD-associated parameter in the subject. The AD-associated parameter may be selected from: Investigators Global Assessment (IGA); Eczema Area and Severity Index (EASI); Scoring atopic dermatitis (SCORAD); and/or pruritus Numeric Rating Scale (NRS).

The IGA is an instrument used in clinical trials to rate the severity of the subject's global AD and is based on a 5-point scale ranging from 0 (clear) to 4 (severe) based on the condition of the disease at the time of evaluation.

morphological

| | Disease | Standard IGA | IGA morphological |
|---|---|---|---|
| Score | severity | scale | descriptors |
| 0 | Clear | No inflammatory | No erythema and no |
| | | signs of atopic | elevation |
| | | dermatitis | (papulation/infiltration). |
| 1 | Almost clear | Just perceptible | Barely perceptible |
| | | erythema, and | erythema and/or minimal |
| | | just perceptible | lesion elevation |
| | | papulation/ | (papulation/infiltration) |
| | | infiltration | that is not widespread. |
| 2 | Mild disease | Mild erythema | Visibly detectable, light |
| | | and mild | pink erythema and very |
| | | papulation/ | slight elevation |
| | | infiltration | (papulation/infiltration). |
| 3 | Moderate | Moderate | Dull red, clearly |
| | disease | erythema and | distinguishable erythema |
| | | moderate | and clearly perceptible but |
| | | papulation/ | not extensive elevation |
| | | infiltration | (papulation/infiltration). |
| 4 | Severe | Severe erythema | Deep/dark red erythema, |
| | disease | and severe | marked and extensive |
| | | papulation/ | elevation |
| | | infiltration | (papulation/infiltration). |

The EASI is a validated measure used in clinical practice and clinical trials to assess the severity and extent of AD (Hanifin et al. “The eczema area and severity index (EASI): assessment of reliability in atopic dermatitis. EASI Evaluator Group. Experimental dermatology” (2001) 10(1):11-18). SCORAD is one of the most commonly used disease severity scores in clinical trials with AD and in clinical practice (see “European Task Force on Atopic Dermatitis. Severity scoring of atopic dermatitis: the SCORAD index. Consensus report of the European task force on atopic dermatitis” Dermatology (1993) 186(1):23-31).

Worst Daily Pruritus NRS is established according to FDA and EMA recommendations (see, e.g. FDA “The Food and Drug Administration. Guidance for Industry. Patient-Reported Outcome Measures: Use in Medical Product Development to Support Labeling Claims. 2009” and EMA “Reflection paper on the regulatory guidance for the use of health-related quality of life (HRQoL) measures in the evaluation of medicinal products. EMEA/CHMP/EWP/139391/2004. 2005). For pruritus NRS, a subject assesses their worst itch severity over the past 24 hours using an 11 point NRS (” Worst Daily Pruritus NRS″) from 0 (no itch) to 10 (worst itch imaginable).

For each AD-associated parameter the improvement or maintained improvement is measured relative to baseline. An improvement in this context can be a reduction in IGA score, a reduction in EASI score, a reduction in SCORAD score (where >50 severe, 25-50 is moderate, <25 is mild), reduction in pruritus NRS score, where each score is compared to baseline.

The baseline is an initial measurement of an AD-associated parameter or patient-related outcome (or any other parameter) that is taken before initiation of treatment by the method described herein, i.e. a measurement taken before the “baseline dose” (defined elsewhere).

An Investigator's Global Assessment 0 or 1 (IGA 0/1; clear or almost clear skin) and/or ≥75% improvement of Eczema Area and Severity Index (EASI-75) are the regulatory primary efficacy endpoints in Phase 3 clinical trials in AD. Thus, the methods described herein may preferably achieve or maintain an Investigator's Global Assessment (IGA) score of 0 or 1 and/or ≥75% improvement of Eczema Area and Severity Index (EASI-75) over baseline (e.g. as shown in Examples 1 and 2 herein). In some embodiments, the methods may achieve or maintain a ≥50% improvement of Eczema Area and Severity Index (EASI-50) over baseline.

Additionally, or alternatively, the methods described herein may improve at least one patient-related outcome (PRO) selected from the group consisting of: worst daily pruritus Numerical Rating Scale (NRS) (see pruritus NRS discussed above), eczema-related sleep interference, Patient Oriented Eczema Measure (POEM), Dermatology Life Quality Index (DLQI), Patient Global Impression of Bother (PGI-B), Hospital Anxiety and Depression Scale (HADS), Short Form (36) Health Survey (SF-36) and EuroQoL 5-Dimension Health Questionnaire 5 Level (EQ-5D-5L).

For eczema-related sleep interference NRS, a subject rates how much their eczema interfered with their sleep the previous night using an 11 point NRS from 0 (no interference) to 10 (complete interference). The POEM is a validated questionnaire used to assess disease symptoms in AD patients in both clinical practice and clinical trials (see Charman et al. “The patient-oriented eczema measure: development and initial validation of a new tool for measuring atopic eczema severity from the patients perspective” Arch Dermatol. (2004) 140(12):1513-1519). DLQI is a patient-reported validated questionnaire with content specific to subjects with dermatology conditions (see Finlay et al. “Dermatology Life Quality Index (DLQI)—a simple practical measure for routine clinical use” Clin Exp Dermatol. (1994) 19(3):210-216). The Patient Global Impression of Bother (PGI-B) is designed to capture the subject's perception of how bothered they have been by their AD over the past 24 hours at the time of completion. A 5-point categorical response scale will be used (‘not at all’, ‘slightly’, ‘somewhat’, ‘a lot’, ‘very much’). The Hospital Anxiety and Depression Scale (HADS) is a Likert-scale tool widely used to detect states of anxiety and depression in a general hospital setting (see Zigmond A S, Snaith R P. “The hospital anxiety and depression scale”. Acta Psychiatr Scand. 1983; 67(6):361-70). The tool consists of 14 items that assess the subject's anxiety (7 items) and depression (7 items) during the last week. Each item is scored from 0 to 3, with high scores indicating more severe anxiety or depression. Short Form (36) Health Survey (SF-36) is a patient-reported survey designed to evaluate health status by generating scores for 8 health domains (physical functioning, role physical, bodily pain, general health, vitality, social functioning, role emotional, and mental health) and 2 psychometrically derived summary scores (a physical component summary and a mental component summary). (see Ware J E J, Sherbourne C D. “The MOS 36-item short-form health survey (SF-36). I. Conceptual framework and item selection” Med Care. 1992; 30(6):473-83). EuroQoL 5-Dimension Health Questionnaire 5 Level (EQ-5D-5L) is a standardised measure of health status developed by the EuroQoL group to provide a simple, generic measure of health for clinical and economic appraisal (see Greiner W et al. “A single European currency for EQ-5D health states. Results from a six-country study” The European journal of health economics: HEPAC: health economics in prevention and care. 2003; 4(3):222-31). The EQ-5D-5L is a self-administered questionnaire used to assess health status ‘today’ and is divided into 2 sections: The first section includes 5 dimensions (mobility, self-care, usual activity, pain/discomfort, and anxiety/depression); each dimension is assessed by the subject using a 5-point scale (‘no problems’, ‘slight problems’, ‘moderate problems’, ‘severe problems’, and ‘extreme problems’). The second section consists of a vertical visual analogue scale anchored at 0 (‘the worst health you can imagine’) and 100 (‘the best health you can imagine’).

The methods may maintain improvement in at least one patient-related outcome (PRO) selected from the group consisting of: worst daily pruritus Numerical Rating Scale (NRS), eczema-related sleep interference, Patient Oriented Eczema Measure (POEM), Dermatology Life Quality Index (DLQI), Patient Global Impression of Bother (PGI-B), Hospital Anxiety and Depression Scale (HADS), Short Form (36) Health Survey (SF-36) and EuroQoL 5-Dimension Health Questionnaire 5 Level (EQ-5D-5L). For each PRO, the improvement or maintained improvement is relative to baseline. An improvement in this context can be a reduction (e.g. a ≥3 point reduction) in the PRO score (or, for example, a ≥4 point reduction for DLQI), where the score is compared to baseline.

In preferred embodiments, the method described herein may achieve: (a) ≥50% improvement of Eczema Area and Severity Index (EASI-50); and/or (b) ≥75% improvement of Eczema Area and Severity Index (EASI-75) (e.g. after 16 weeks or after 26 weeks, as illustrated in Examples 1 and 2).

### TCS Dependence

Long-term application of a TCS is not recommended because of the risk of skin atrophy, dyspigmentation, acneiform eruptions, and risks associated with systemic absorption (e.g. hypothalamic pituitary axis effects, Cushing's disease, etc.). Repeated application of any topical therapy over a long period of time or to large surface areas can also lead to reduced patient compliance.

The methods described herein may reduce the topical corticosteroid (TCS) dependence of the subject with AD (especially moderate-to-severe or severe AD).

Reduced dependence may be assessed by comparing the cumulative amount (in grams) of a formulation containing TCS applied by a subject after initiation of a method described herein over a particular time interval (e.g. 16 weeks or 26 weeks), as compared to a placebo-treated subject. For example, a subject may use at least 0.2 g less, at least 0.3 g less, at least 0.4 g less or at least 0.5 g less TCS per day, as compared to a placebo-treated subject. Typically, a subject may use at least 0.5 g less TCS per day, as compared to a placebo-treated subject.

Reduced dependence may also be assessed by the number of TCS-free days (which may still include lower potency TCS and TCI) after initiation of the method, as compared to the same measurement performed at baseline.

A TCS can be classified as group I, group II, group III and group IV topical corticosteroid. According to the Anatomical Therapeutic Classification System of World Health Organization, corticosteroids are classified as weak/lower potency (group I), moderately potent (group II) and potent (group III) and very potent (group IV), based on their activity as compared to hydrocortisone. Group IV TCS (very potent) are up to 600 times as potent as hydrocortisone and include clobetasol propionate and halcinonide. Group III TCS (potent) are 50 to 100 times as potent as hydrocortisone and include betamethasone valerate, betamethasone dipropionate, diflucortolone valerate, hydrocortisone-17-butyrate, mometasone furoate, and methylprednisolone aceponate. Group II TCS (moderately potent) are 2 to 25 times as potent as hydrocortisone and include clobetasone butyrate, and triamcinolone acetonide. Group I TCS (mild) includes hydrocortisone.

The term “TCS-free day” means a day in which the subject does not use a TCS of Group II, Group III or Group IV.

For example, the subject the number of TCS-free days may increase by about 0.5 day, about 0.75 day, about 1 day, about 1.5 days, about 2 days, about 3 days or more averaged over a week, as compared to a placebo-treated subject. Typically, the number of TCS-free days may increase by about 0.5 day, as compared to a placebo-treated subject.

In some embodiments, the IL-13 binding protein can be administered as a monotherapy e.g. without TCS. In other embodiments, the IL-13 binding protein is administered in combination with a second therapeutic agent selected from the group consisting of a TCS, a topical calcineurin inhibitor, an anti-histamine, an emollient, or an anti-bacterial therapeutic. In preferred embodiments, the IL-13 binding protein is administered in combination with a TCS.

The IL-13 binding protein may be used in combination with a TCS so as to wean a subject off TCS use. Accordingly, in the methods described herein a medium-potency or high-potency TCS may be administered alongside the IL-13 binding protein. The amount of TCS can then be reduced by at least 20%, at least 30%, at least 40%, at least 50%, at least 60%, at least 70%, at least 80%, at least 90%, at least 95%, or around 100% after initiation of the method (e.g. over a 3-4 month period), as compared to the amount of TCS at baseline.

### IL-13 Binding Protein

An IL-13 binding protein is a protein that specifically binds to and neutralizes human IL-13.

Herein, the term “specifically binds” means that a protein (such as an antibody or antigen-binding fragment thereof) forms a complex with an antigen that is relatively stable under physiological conditions. Methods for determining whether a protein specifically binds to an antigen are well known in the art and include, for example, equilibrium dialysis, surface plasmon resonance (e.g. using a BIAcore 200 Biosensor (BIAcore AB), and the like. For example, an IL-13 binding protein (e.g. an anti-IL-13 antibody or IL-13 binding fragment thereof) that “specifically binds” IL-13 may bind IL-13 with a Kp of less than about 1000 nM, less than about 500 nM, less than about 100 nM, less than about 50 nM, less than about 20 nM, less than about 10 nM, less than about 5 nM, less than about 1 nM, less than about 0.5 nM, less than about 0.25 nM, less than about 0.1 nM or less than about 0.05 nM, as measured by surface plasmon resonance at 25° C. The exemplified antibody, tralokinumab binds human bound human IL-13 with a K_{D} of 178 pM, as measured by surface plasmon resonance (see WO 2005/007699 for detailed methods). Accordingly, in a preferred embodiment, the anti-IL-13 antibody has a K_{D} of less than about 200 pM, as measured by surface plasmon resonance at 37° C. or 25° C. Although an IL-13 binding protein specifically binds human IL-13, it may have cross-reactivity to other antigens, such as IL-13 from other (non-human) species.

Methods for measuring neutralisation activity are well known in the art. Neutralisation activity can be measured in an IL-13 dependent TF-1 cell proliferation assay relative to a control antibody that is not directed to IL-13, as described in WO 2005/007699. In this assay, inhibition of IL-13 dependent proliferation is determined by measuring the reduction in incorporation of tritiated thymidine into the newly synthesized DNA of dividing cells. Briefly, commercial TF-1 cells are maintained according to supplied protocols. Assay media comprises RPMI-1640 with GLUTAMAX I (Invitrogen) containing 5% FBS and 1% sodium pyruvate. Prior to each assay, TF-1 cells are pelleted by centrifugation at 300×g for 5 minutes, the media removed by aspiration and the cells resuspended in assay media. This process is repeated twice with cells resuspended at a final concentration of 10⁵ cells/mL in assay media. Test solutions of antibody (in triplicate) are diluted to the desired concentration in assay media. An antibody that is not directed at IL-13 is used as a negative control. Recombinant bacterially derived human or murine IL-13 is added to a final concentration of 50 ng/mL when mixed with the appropriate test antibody in a total volume of 100 μL/well in a 96 well assay plate. The concentration of IL-13 used in the assay is selected as the dose that at final assay concentration gives approximately 80% of the maximal proliferative response. All samples are incubated for 30 minutes at room temperature. 100 μL of resuspended cells are then added to each assay point to give a total assay volume of 200 μL/well. Assay plates are incubated for 72 hours at 37° C. under 5% CO₂. 25 μL of tritiated thymidine (10 μCi/mL) is then added to each assay point and assay plates are returned to the incubator for a further 4 hours. Cells are harvested on glass fibre filter plates (Perkin Elmer) using a cell harvester. Thymidine incorporation is determined using a Packard TopCount microplate liquid scintillation counter.

### Anti-IL-13 Antibodies and IL-13-Binding Thereof

Typically, the IL-13 binding protein is an anti-IL-13 antibody or an IL-13-binding fragment thereof.

The term “antibody”, as used herein, includes immunoglobulin molecules comprising four polypeptide chains, two heavy (H) chains and two light (L) chains inter-connected by disulfide bonds, as well as multimers thereof (e.g. IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some cases, the FRs of the anti-IL-13 antibody (or IL-13-binding fragment or derivative thereof) may be identical to the human germline sequences, or may be naturally or artificially modified.

The heavy chain constant region of the antibodies may be from any types of constant region, such as IgG, IgM, IgD, IgA, and IgE. Generally, the antibody is an IgG (e.g. isotype IgG1, IgG2, IgG3 or IgG4). Preferably, the antibody is an IgG4, as exemplified herein.

The antibody may be a mouse, human, primate, humanized or chimeric antibody. The antibody may be polyclonal or monoclonal. For therapeutic applications, monoclonal and human (or humanized) antibodies are preferred. In a particularly preferred embodiment, the antibody is human or humanized, and monoclonal.

The antibody can be a multispecific (e.g. bispecific) antibody. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen binding fragment of an antibody as described herein using routine techniques available in the art. For example, the methods that use of bispecific antibodies, wherein one arm of an immunoglobulin is specific for IL-13, and the other arm of the immunoglobulin is specific for a second therapeutic target or is conjugated to a therapeutic moiety.

An IL-13-binding fragment of an anti-IL-13 antibody may be any naturally occurring, enzymatically obtainable, synthetic, or genetically engineered polypeptide. Such fragments may be derived, e.g. from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g. commercial sources, DNA libraries (including, e.g. phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of IL-13-binding fragments include: Fab, Fab′, F(ab′)2, Fd, Fv, single-chain Fv (scFv), disulphide-linked Fvs, dAb fragments, and other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains.

An IL-13-binding fragment of an anti-IL-13-binding antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

The anti-IL-13 antibody, or an IL-13-binding fragment thereof, may comprise: a heavy chain complementarity determining region 1 (HCDR1) comprising an amino acid sequence of SEQ ID NO:1; a heavy chain complementarity determining region 2 (HCDR2) comprising an amino acid sequence of SEQ ID NO:2; a heavy chain complementarity determining region 3 (HCDR3) comprising an amino acid sequence of SEQ ID NO:3; a light chain complementarity determining region 1 (LCDR1) comprising an amino acid sequence of SEQ ID NO:4; a light chain complementarity determining region 2 (LCDR2) comprising an amino acid sequence of SEQ ID NO:5; and a light chain complementarity determining region 3 (LCDR3) comprising an amino acid sequence of SEQ ID NO:6. The anti-IL-13 antibody, or an IL-13-binding fragment thereof, may comprise a heavy chain variable region (HCVR) and a light chain variable region (LCVR), wherein: (i) the heavy chain variable region comprises: a heavy chain complementarity determining region 1 (HCDR1) comprising an amino acid sequence of SEQ ID NO:1; a heavy chain complementarity determining region 2 (HCDR2) comprising an amino acid sequence of SEQ ID NO:2; and a heavy chain complementarity determining region 3 (HCDR3) comprising an amino acid sequence of SEQ ID NO:3; and (ii) the light chain variable region comprises: a light chain complementarity determining region 1 (LCDR1) comprising an amino acid sequence of SEQ ID NO:4; a light chain complementarity determining region 2 (LCDR2) comprising an amino acid sequence of SEQ ID NO:5; and a light chain complementarity determining region 3 (LCDR3) comprising an amino acid sequence of SEQ ID NO:6. In addition, the anti-IL-13 antibody, or an IL-13-binding fragment thereof, may further comprise: (i) an amino acid sequence that is at least 80%, at least 85%, at least 90%, at least 95%, at least 96%, at least 97%, at least 98%, or at least 99% identical to a heavy chain variable region sequence of SEQ ID NO:8; and/or (ii) an amino acid sequence that is 80%, at least 85%, at least 90%, at least 95%, at least 96%, at least 97%, at least 98%, or at least 99% identical to a light chain variable region sequence of SEQ ID NO:10. The anti-IL-13 antibody, or an IL-13-binding fragment thereof, may comprise a heavy chain variable region sequence of SEQ ID NO:8 and a light chain variable region sequence of SEQ ID NO:10.

The anti-IL-13 antibody, or the IL-13-binding fragment thereof, may comprise: (i) an amino acid sequence that is at least 80%, at least 85%, at least 90%, at least 95%, at least 96%, at least 97%, at least 98%, or at least 99% identical to the heavy chain sequence of SEQ ID NO:11; and/or (ii) an amino acid sequence that is at least 80%, at least 85%, at least 90%, at least 95%, at least 96%, at least 97%, at least 98%, or at least 99% identical to the light chain sequence of SEQ ID NO:12. In some cases, the anti-IL-13 antibody, or an IL-13-binding fragment or IL-13-binding derivative thereof, comprises a heavy chain of SEQ ID NO:11 and a light chain sequence of SEQ ID NO:12.

One such antibody that can be used in the methods described herein is the anti-IL-13 antibody, tralokinumab (as described in the “International Nonproprietary Names for Pharmaceutical Substances (INN)” list 102 (WHO Drug Information (2009) 23(4): pp 348)). Tralokinumab is a fully human IgG4-lambda antibody, which specifically binds and neutralises human IL-13.

TABLE 1

| | | |
|---|---|---|
| SEQ ID | | |
| number | Name | Sequence |

Tralokinumab

| | | |
|---|---|---|
| SEQ ID | HCDR1 | NYGLS |
| NO: 1 | | |
| SEQ ID | HCDR2 | WISANNGDTNYGQEFQG |
| NO: 2 | | |
| SEQ ID | HCDR3 | DSSSSWARWFFDL |
| NO: 3 | | |
| SEQ ID | LCDR1 | GGNIIGSKLVH |
| NO: 4 | | |
| SEQ ID | LCDR2 | DDGDRPS |
| NO: 5 | | |
| SEQ ID | LCDR3 | QVWDTGSDPVV |
| NO: 6 | | |
| SEQ ID | cDNA | caggtccagctggtgcagtc |
| NO: 7 | heavy | tggggctgaggtgaagaagc |
| | chain | ctggggcctcagtgaaggtc |
| | variable | tcctgcaaggcttctggtta |
| | domain | cacctttacaaattatggtc |
| | | tcagctgggtgcgacaggcc |
| | | cctggacaagggcttgagtg |
| | | gatgggatggatcagcgcta |
| | | ataatggcgacacaaattat |
| | | ggacaggaattccagggcag |
| | | agtcaccatgaccacagata |
| | | catccacgagcacagcctac |
| | | atggagttgaggagcctgag |
| | | atctgacgacacggccgttt |
| | | attactgtgcgagagactcc |
| | | agcagcagctgggcccgctg |
| | | gtttttcgatctctggggcc |
| | | gggggacactggtcaccgtc |
| | | tcctca |
| SEQ ID | polypeptide | QVQLVQSGAEVKKPGASVKV |
| NO: 8 | sequence | SCKASGYTFTNYGLSWVRQA |
| | heavy | PGQGLEWMGWISANNGDTNY |
| | chain | GQEFQGRVTMTTDTSTSTAY |
| | variable | MELRSLRSDDTAVYYCARDS |
| | region | SSSWARWFFDLWGRGTLVTV |
| | | SS |
| SEQ ID | cDNA light | tcctatgtgctgactcagcc |
| NO: 9 | chain | accctcggtgtcagtggccc |
| | variable | caggaaagacggccaggatt |
| | domain | acctgtgggggaaacatcat |
| | | tggaagtaaacttgtacact |
| | | ggtaccagcagaagccaggc |
| | | caggcccctgtgctggtcat |
| | | ctatgatgatggcgaccggc |
| | | cctcagggatccctgagcga |
| | | ttctctggctccaactctgg |
| | | gaacacggccaccctgacca |
| | | tcagcagggtcgaggccggg |
| | | gatgaggccgactattattg |
| | | tcaggtgtgggatactggta |
| | | gtgatcccgtggtattcggc |
| | | ggagggaccaagctgaccgt |
| | | cctaggt |
| SEQ ID | polypeptide | SYVLTQPPSVSVAPGKTARI |
| NO: 10 | sequence light | TCGGNIIGSKLVHWYQQKPG |
| | chain variable | QAPVLVIYDDGDRPSGIPER |
| | region | FSGSNSGNTATLTISRVEAG |
| | | DEADYYCQVWDTGSDPVVFG |
| | | GGTKLTVL |
| SEQ ID | Heavy chain | QVQLVQSGAEVKKPGASVKV |
| NO: 11 | | SCKASGYTFTNYGLSWVRQA |
| | | PGQGLEWMGWISANNGDTNY |
| | | GQEFQGRVTMTTDTSTSTAY |
| | | MELRSLRSDDTAVYYCARDS |
| | | SSSWARWFFDLWGRGTLVTV |
| | | SSASTKGPSVFPLAPCSRST |
| | | SESTAALGCLVKDYFPEPVT |
| | | VSWNSGALTSGVHTFPAVLQ |
| | | SSGLYSLSSVVTVPSSSLGT |
| | | KTYTCNVDHKPSNTKVDKRV |
| | | ESKYGPPCPSCPAPEFLGGP |
| | | SVFLFPPKPKDTLMISRTPE |
| | | VTCVVVDVSQEDPEVQFNWY |
| | | VDGVEVHNAKTKPREEQFNS |
| | | TYRVVSVLTVLHQDWLNGKE |
| | | YKCKVSNKGLPSSIEKTISK |
| | | AKGQPREPQVYTLPPSQEEM |
| | | TKNQVSLTCLVKGFYPSDIA |
| | | VEWESNGQPENNYKTTPPVL |
| | | DSDGSFFLYSRLTVDKSRWQ |
| | | EGNVFSCSVMHEALHNHYTQ |
| | | KSLSLSLGK |
| SEQ ID | Light chain | SYVLTQPPSVSVAPGKTARI |
| NO: 12 | | TCGGNIIGSKLVHWYQQKPG |
| | | QAPVLVIYDDGDRPSGIPER |
| | | FSGSNSGNTATLTISRVEAG |
| | | DEADYYCQVWDTGSDPVVFG |
| | | GGTKLTVLGQPKAAPSVTLF |
| | | PPSSEELQANKATLVCLISD |
| | | FYPGAVTVAWKADSSPVKAG |
| | | VETTTPSKQSNNKYAASSYL |
| | | SLTPEQWKSHRSYSCQVTHE |
| | | GSTVEKTVAPTECS |

Methods for identifying, isolating and testing (e.g. binding and neutralisation) of antibodies and fragment thereof are well-known in the art. See WO 2005/007699, which teaches the identification and characterisation of various anti-IL13 antibodies and fragments and provides suitable methods for doing so.

### Dose and Dosing Regimen

The invention provides an interleukin-13 (IL-13) binding protein as described above (e.g. an anti-IL-13 antibody or IL-13 binding fragment thereof) for use in any method of treatment described herein, wherein the method comprises the steps of: (a) administering a first dose of the IL-13 binding protein to the subject; and (b) administering one or more secondary dose(s) of the IL-13 binding protein to the subject, wherein each secondary dose is administered to the subject from 12 days to 35 days after the immediately preceding dose. Preferably, each secondary dose is administered to the subject from 12 days to 16 days after the immediately preceding dose, e.g. 14 days after the immediately preceding dose, or from 25 days to 31 days after the immediately preceding dose, e.g. about 4 weeks after the immediately preceding dose.

The term “dose” refers to the amount (mass) of IL-13 binding protein administered to the subject on the particular treatment day. For example, a dose of 300 mg of IL-13 binding protein means that on a treatment day a total of 300 mg of IL-13 binding protein is given to the subject. In some embodiments, a dose is administered in a single administration step (e.g. one injection). However, in some embodiments, one, two, three, four or more administration steps (e.g. one, two, three, four or more injections) may be used to provide the subject with the desired dose.

The terms “prior dose”, “first dose”, “secondary dose”, and “tertiary dose” refer to the temporal sequence of administration of the IL-13 binding protein. The term “first dose” is a single dose of IL-13 binding protein that is followed by one or more secondary dose(s). The first dose may be preceded by one or more prior dose(s), or the “first dose” may be the initiation of treatment by the method described herein (in the latter case, this dose can therefore be referred to as the “baseline dose”). Subsequent to the first dose is one or more secondary dose(s); and the one or more secondary dose(s) may be followed by one or more tertiary dose(s).

The phrase “immediately preceding dose” means, in a sequence of multiple doses, the dose of IL-13 binding protein which is administered to a patient prior to the administration of the very next dose in the sequence, with no intervening doses of the IL-13 binding protein.

“Dosing frequency” is the frequency of administering a dose of the IL-13 binding protein. Thus, a decrease in dosing frequency means an increase in the time interval between doses. Common terminology used in relation to dosing frequency is QW (once weekly), Q2W (once every 2 weeks), Q3W (every 3 weeks), or Q4W (every 4 weeks).

The first dose may be from about 10 mg to about 600 mg of the IL-13 binding protein, from about 50 mg to 500 mg, from about 100 mg to about 400 mg, from about 250 mg to about 350 mg or from about 280 mg to about 320 mg of IL-13 binding protein. For example, the first dose is about 10 mg, about 25 mg, about 50 mg, about 100 mg, about 150 mg, about 200 mg, about 250 mg, about 300 mg, about 400 mg, about 500 mg or about 600 mg. In some cases, the first dose is 600 mg or less, 500 mg or less, 400 mg or less, 300 mg or less, 200 mg or less, or 200 mg or less. In preferred embodiments, the first dose is about 600 mg of IL-13 binding protein (e.g. as illustrated in the examples).

Each secondary dose may be administered to the subject from 12 days to 35 days, from 12 days to 30 days, from 12 days to 25 days, from 12 days to 20 days, from 12 days to 16 days, from 12 days to 15 days, from 12 days to 14 days, from 18 days to 35 days, from 21 days to 35 days, from 22 days to 34 days, from 24 days to 32 days, from 25 days to 31 days, from 26 days to 30 days, or from 27 days to 29 days after the immediately preceding dose. In certain cases, each secondary dose may be administered to the subject about 14 days (i.e. 2 weeks) after the immediately preceding dose (as exemplified herein).

In the methods described herein, the method may be carried out until it provides improvement in an AD-associated parameter and/or patient-related outcome as described herein. In some cases, the method may provide an improvement in an AD-associated parameter and/or patient-related outcome in around 2 weeks, around 3 weeks, around 12 weeks, around 3 months, around 16 weeks, around 24 weeks, around 26 weeks, around 6 months, around 32 weeks, around 36 weeks, around a year, around 52 weeks or more than 52 weeks. In some preferred embodiments, the improvement in an AD-associated parameter and/or patient-related outcome is provided in around 16 weeks or 26 weeks.

In some cases, the method may be continued until the subject reaches a low disease state. For example, the subject may reach a low disease state in around 4 weeks, around 8 weeks, around 12 weeks, around 3 months, around 16 weeks, around 24 weeks, around 26 weeks, around 6 months, around 32 weeks, around 36 weeks, around a year, around 52 weeks or more than 52 weeks. In some preferred embodiments, the subject may reach a low disease state in around 16 weeks or 26 weeks.

In some cases, the method may be carried out for at least 4 weeks, at least 8 weeks, at least 12 weeks, at least 3 months, at least 16 weeks, at least 24 weeks, at least 26 weeks, at least 6 months, at least 32 weeks, at least 36 weeks, at least a year, or at least 52 weeks or more. In some cases, the method may be carried out for around 2 weeks, around 3 weeks, around 12 weeks, around 3 months, around 16 weeks, around 24 weeks, around 6 months, around 32 weeks, around 36 weeks, around a year, around 52 weeks. In preferred embodiments, the method is carried out for at least 26 weeks.

Herein, the phrase “low disease state” is an Investigator's Global Assessment (IGA) score of 0 or 1 and/or ≥75% improvement of Eczema Area and Severity Index (EASI-75) over baseline.

Step (b) of the method (i.e. administering one or more secondary dose(s) of the IL-13 binding protein to the subject) may be continued (i.e. by administering more than one secondary dose) for from 8 weeks to 52 weeks, from 12 to 40 weeks or from 16 to 36 weeks. The one or more secondary dose(s) may be administered for at least 8 weeks, at least 12 weeks, at least 3 months, at least 16 weeks, at least 20 weeks, at least 24 weeks, at least 6 months, at least 28 weeks, at least 32 weeks, at least 36 weeks, at least a year, at least 52 weeks or more. Step (b) of the method may be continued (i.e. by administering more than one secondary dose) for around 8 weeks, around 12 weeks, around 3 months, around 16 weeks, around 20 weeks, around 24 weeks, around 6 months, around 28 weeks, around 32 weeks, around 36 weeks, around a year, around 52 weeks or more. In some embodiments, the one or more secondary dose(s) is administered for at least 16 weeks, for at least 24 weeks or for at least 26 weeks. Additionally, or alternatively, step (b) may be continued until the method provides improvement in an AD-associated parameter and/or patient-related outcome as described herein. Step (b) of may be continued to maintain improvement in an AD-associated parameter and/or patient-related outcome as described herein. In particular cases, step (b) may be continued until the subject reaches a low disease state.

Each secondary dose may be from about 10 mg to about 600 mg of the IL-13 binding protein, from about 50 mg to 500 mg, from about 100 mg to about 400 mg, from about 250 mg to about 350 mg or from about 280 mg to about 320 mg of IL-13 binding protein. For example, each secondary dose is about 10 mg, about 25 mg, about 50 mg, about 100 mg, about 150 mg, about 200 mg, about 250 mg, about 300 mg, about 400 mg or about 500 mg. In some cases, each secondary dose is 600 mg or less, 500 mg or less, 400 mg or less, 300 mg or less, 200 mg or less or 200 mg or less. In preferred embodiments, each secondary dose is about 300 mg of IL-13 binding protein (e.g. as in the examples). In some embodiments, the first dose and one or more secondary dose(s) are the same amount (i.e. in milligrams) of IL-13 binding protein. In other embodiments, the first dose is greater than the one or more secondary doses. For example, in some embodiments the first dose is 600 mg and the one or more secondary doses is 300 mg.

In preferred embodiments, the method comprises the steps of: (a) administering a first dose of about 600 mg of the IL 13 binding protein (e.g. tralokinumab) to the subject; and (b) administering one or more secondary dose(s) of about 300 mg of the IL-13 binding protein (e.g. tralokinumab) to the subject, wherein each secondary dose is administered to the subject about 2 weeks after the immediately preceding dose, optionally wherein the method is carried out for about 26 weeks. Preferably, each administration is by subcutaneous injection.

In some embodiments the first dose is a bolus dose which is double the amount of the doses following the bolus dose. In some embodiments the first dose is a 600 mg dose and the dose(s) following the 600 mg dose is/are 300 mg dose(s). The bolus dose is typically twice the amount of the dose administered with the next administration. For example, a dose of 600 mg is used as a bolus dose when the next dose administered is 300 mg.

### Administration

In the methods described herein, the IL-13 binding protein (e.g. an anti-IL-13 antibody or an IL-13-binding fragment thereof) may be administered by any appropriate method. Typically, administration is parenteral, e.g. intradermal, intramuscular, intravenous and subcutaneous. Subcutaneous administration is particularly preferred (e.g. as illustrated in the examples). Each dose of the IL-13 binding protein may therefore be administered subcutaneously.

Administration is preferably in a “therapeutically effective amount”, this being sufficient to show improvement or maintained improvement in one or more AD-associated parameter or patient-related outcome as described herein, or achievement of a low disease state.

Administration may be by any convenient route, for example by infusion or bolus injection, by absorption through epithelial or mucocutaneous linings (e.g. oral mucosa, rectal and intestinal mucosa, etc.).

Subcutaneous or intravenous delivery may be with a standard needle and syringe (e.g. including with a prefilled syringe). It is envisaged that the methods described herein will not be restricted to use in the clinic. Therefore, subcutaneous injection using a needle free device is also preferred. Such delivery devices can be reusable or disposable. Numerous reusable pen and autoinjector delivery devices are known in the art and may find use in the present invention. Examples include AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ 1, 11 and 111 (Nova Nordisk, Copenhagen, Denmark), NOVOPEN JUNIOR™ (Nova Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (Sanofi-Aventis, Frankfurt, Germany). Exemplary disposable pen delivery devices for subcutaneous delivery that may find use in the present invention applications include the SOLOSTAR™ pen (Sanofi-Aventis), the FLEXPEN™ (Nova Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL).

Each dose of IL-13 binding protein is not necessarily administered in a single administration step (e.g. one injection or one tablet etc.). Indeed, depending on the concentration of the IL-13 binding protein (e.g. in the pharmaceutical composition), one, two, three, four or more administration steps (e.g. one, two, three, four or more injections) may be required to provide the subject with the required amount of IL-13 binding protein (e.g. a 300 mg dose, for example). Thus, in some embodiments, each dose of the IL-13 binding protein is administered in two or four injections (e.g. subcutaneously). In some embodiments, each injection provides 150 mg IL-13 binding protein and two injections are required for a 300 mg dose and four injections are required for a 600 mg dose. Typically subcutaneous injections have a volume of around 1.5 mL or less, such as a volume of from 0.2 to 1.5 mL, e.g. around 1 mL.

### Monotherapy and Combination Therapy

The methods described herein may be a monotherapy. As used herein, the term “monotherapy” is a therapy which uses a single drug to treat a disease or condition. Therefore, a subject that is treated with a monotherapy will receive only a single drug to treat the relevant disorder, i.e. AD. For example, an anti-IL-13 antibody monotherapy refers to a monotherapy which comprises the administration of anti-IL-13 antibody to the subject as the sole drug for the treatment of AD.

The methods described herein may be a combination therapy. As used herein, the term “combination therapy” is a therapy which uses more than one drug to treat a disease or condition. For example, a subject that is treated with a combination therapy will receive more than one drug (e.g. two, three or more) to treat AD.

In some embodiments, the IL-13 binding protein is administered in combination with a topical therapy (such as a topical corticosteroid or a topical calcineurin inhibitor). In some instances, the additional treatment (e.g. TCS or TCI) is administered as needed by the subject.

In some cases, the IL-13 binding protein is administered in combination with a second therapeutic agent selected from the group consisting of a topical corticosteroid, a topical calcineurin inhibitor, an anti-histamine, an emollient, or an anti-bacterial therapeutic. In some cases, the IL-13 binding protein is administered in combination with a Group I, Group II, Group III or Group IV corticosteroid. Preferably, the IL-13 binding protein can be administered in combination with mometasone furoate (e.g. 0.1% cream).

### Pharmaceutical Compositions and Formulations

The present invention envisages methods where each dose of the IL-13 binding protein (e.g. an anti-IL-13 antibody or an IL-13-binding fragment thereof) is administered as a pharmaceutical composition.

The pharmaceutical compositions may be formulated with suitable carriers, excipients, and other agents that provide suitable transfer, delivery, tolerance, and the like. A multitude of formulations can be found in the formulary known to all pharmaceutical chemists: Remington's Pharmaceutical Sciences, Mack Publishing Company, Easton, PA.

The dose administered to a patient according to the methods described herein may be varied depending upon the age and the size of the patient, symptoms, conditions, route of administration, and the like. The dose can be calculated according to body weight or body surface area.

Thus, the pharmaceutical compositions may comprise, in addition to the active ingredient (i.e. the IL-13 binding protein), a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous or subcutaneous. Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection or subcutaneous injection, the pharmaceutical composition may be a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The pharmaceutical composition may be a liquid formulation or a lyophilized formulation which is reconstituted before use. As excipients for a lyophilized formulation, for example, sugar alcohols, or saccharides (e.g. mannitol or glucose) may be used. In the case of a liquid formulation, the pharmaceutical composition is usually provided in the form of containers with defined volume, including sealed and sterilized plastic or glass vials, ampoules and syringes, as well as in the form of large volume containers like bottles. Preferably, in the methods described herein, the pharmaceutical composition is a liquid formulation.

Exemplary pharmaceutical compositions that can be used in the context of the present invention are disclosed in, for example, WO 2007/036745 and WO 2018/158332.

Preferably, the IL-13 binding protein may be present within the pharmaceutical composition at a concentration of from 1 mg/mL to 200 mg/mL, more preferably 150 mg/mL.

Preferably, the pharmaceutical composition may be buffered to a pH of 5.2 to 5.7, most preferably 5.5 (e.g. ±0.1). The selection of such a pH confers significant stability to the pharmaceutical composition. Examples of alternative buffers that control the pH in this range include succinate, gluconate, histidine, citrate, phosphate, glutarate, cacodylate, sodium hydrogen maleate, tris(hydroxymethyl)aminomethane (Tris), 2-(N-morpholino)ethanesulphonic acid (MES), imidazole. Preferably, the buffer is acetate buffer, more preferably sodium acetate buffer.

Preferably, the acetate buffer is present within the pharmaceutical composition in an amount of from 1 mM to 100 mM, more preferably from 30 mM to 70 mM, especially 50 mM.

It will be appreciated that references to “pharmaceutically acceptable excipient” includes references to any excipient conventionally used in pharmaceutical compositions. Such excipients may typically include one or more surfactant, inorganic or organic salt, stabilizer, diluent, solubilizer, reducing agent, antioxidant, chelating agent, preservative and the like.

Examples of a typical surfactant include: nonionic surfactants (HLB 6 to 18) such as sorbitan fatty acid esters (e.g. sorbitan monocaprylate, sorbitan monolaurate, sorbitan monopalmitate), glycerine fatty acid esters (e.g. glycerine monocaprylate, glycerine monomyristate, glycerine monostearate), polyglycerine fatty acid esters (e.g. decaglyceryl monostearate, decaglyceryl distearate, decaglyceryl monolinoleate), polyoxyethylene sorbitan fatty acid esters (e.g. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g. polyoxyethylene sorbitol tetrastearate, polyoxyethylene sorbitol tetraoleate), polyoxyethylene glycerine fatty acid esters (e.g. polyoxyethylene glyceryl monostearate), polyethylene glycol fatty acid esters (e.g. polyethylene glycol distearate), polyoxyethylene alkyl ethers (e.g. polyoxyethylene lauryl ether), polyoxyethylene polyoxypropylene alkyl ethers (e.g. polyoxyethylene polyoxypropylene glycol ether, polyoxyethylene polyoxypropylene propyl ether, polyoxyethylene polyoxypropylene cetyl ether), polyoxyethylene alkylphenyl ethers (e.g. polyoxyethylene nonylphenyl ether), polyoxyethylene hydrogenated castor oils (e.g. polyoxyethylene castor oil, polyoxyethylene hydrogenated castor oil), polyoxyethylene beeswax derivatives (e.g. polyoxyethylene sorbitol beeswax), polyoxyethylene lanolin derivatives (e.g. polyoxyethylene lanolin), and polyoxyethylene fatty acid amides (e.g. polyoxyethylene stearyl amide); anionic surfactants such as C10-C18 alkyl sulfates salts (e.g. sodium cetyl sulfate, sodium lauryl sulfate, sodium oleyl sulfate), polyoxyethylene C10-C18 alkyl ether sulfates salts with an average of 2 to 4 moles of ethylene oxide (e.g. sodium polyoxyethylene lauryl sulfate), and C8-C18 alkyl sulfosuccinate ester salts (e.g. sodium lauryl sulfosuccinate ester); and natural surfactants such as lecithin, glycerophospholipid, sphingophospholipids (e.g. sphingomyelin), and sucrose esters of C12-C18 fatty acids. The surfactant may be selected from polyoxyethylene sorbitan fatty acid esters. Preferred surfactants are polysorbate 20, 21, 40, 60, 65, 80, 81 and 85, most preferably polysorbate 20 and 80, especially polysorbate 80.

Preferably, the surfactant is present within the pharmaceutical composition in an amount of from 0.001% to 0.1% (w/w), more preferably 0.005% and 0.05% (w/w), especially 0.01% (w/w).

Examples of a typical inorganic salt include: sodium chloride, potassium chloride, calcium chloride, sodium phosphate, sodium sulphate, ammonium sulphate, potassium phosphate and sodium bicarbonate or any other sodium, potassium or calcium salt. Preferably, the inorganic salt is sodium chloride.

Preferably, the inorganic salt is present within the pharmaceutical composition in an amount of from 10 mM to 200 mM, more preferably from 60 mM to 130 mM, especially 85 mM.

Examples of a reducing agent include N-acetylcysteine, Nacetylhomocysteine, thioctic acid, thiodiglycol, thioethanolamine, thioglycerol, thiosorbitol, thioglycolic acid and a salt thereof, sodium thiosulfate, glutathione, and a Cl-C7 thioalkanoic acid.

Examples of an antioxidant include erythorbic acid, dibutylhydroxytoluene, butylhydroxyanisole, alpha-tocopherol, tocopherol acetate, L-ascorbic acid and a salt thereof, L-ascorbic acid palmitate, L-ascorbic acid stearate, sodium bisulfite, sodium sulfite, triamyl gallate and propyl gallate.

Examples of a chelating agent include disodium ethylenediaminetetraacetate (EDTA), sodium pyrophosphate and sodium metaphosphate.

Examples of a stabiliser include creatinine, an amino acid selected from histidine, alanine, glutamic acid, glycine, leucine, phenylalanine, methionine, isoleucine, proline, aspartic acid, arginine, lysine and threonine, a carbohydrate selected from sucrose, trehalose, sorbitol, xylitol and mannose, surfactants selected from polyethylene glycol (PEG; e.g. PEG3350 or PEG 4000) or polyoxyethylene sorbitan fatty acid esters (e.g. polysorbate 20 or polysorbate 80), or any combination thereof.

In one preferred embodiment the stabiliser comprises a single carbohydrate (e.g. trehalose).

In an alternatively preferred embodiment the stabilizer comprises an amino acid in combination with a carbohydrate (e.g. trehalose and alanine or trehalose, alanine and glycine).

In a further alternatively preferred embodiment the stabiliser comprises an amino acid in combination with a carbohydrate and a surfactant (e.g. trehalose, alanine and PEG3350; trehalose, proline and PEG3350; trehalose, alanine and polysorbate 80; trehalose, proline and polysorbate 80; trehalose, alanine, glycine and PEG3350; trehalose, alanine, glycine and polysorbate 80).

In a yet further alternatively preferred embodiment the stabiliser comprises an amino acid in combination with a surfactant (e.g. alanine and PEG3350 or alanine, glycine and PEG3350).

In a yet further alternatively preferred embodiment the stabiliser comprises a carbohydrate in combination with a surfactant (e.g. trehalose and PEG3350 or trehalose and polysorbate 80).

Examples of a preservative include octadecyldimethylbenzyl ammonium chloride, hexamethonium chloride, benzalkonium chloride (a mixture of alkylbenzyldimethylammonium chlorides in which the alkyl groups are long chain compounds), benzethonium chloride, aromatic alcohols such as phenol, butyl and benzyl alcohol, alkyl parabens such as methyl or propyl paraben, catechol, resorcinol, cyclohexanol, 3-pentanol, and m-cresol. In a preferred embodiment, the pharmaceutical composition comprises an IL-13 binding protein as described herein, a surfactant and an inorganic salt buffered to a pH of 5.5±0.1 with acetate buffer.

In a further preferred embodiment, the pharmaceutical composition comprises an IL-13 binding protein as described herein, sodium chloride and polysorbate 80, buffered to a pH of 5.5±0.1 with sodium acetate buffer.

In a yet further preferred embodiment, the pharmaceutical composition comprises an IL-13 binding protein as described herein (e.g. tralokinumab), 50 mM sodium acetate buffer, 85 mM sodium chloride, 0.01% (w/v) polysorbate 80, wherein the pharmaceutical composition has a pH of 5.5.

In a yet further preferred embodiment, the pharmaceutical composition comprises 150 mg/mL of an IL-13 antibody (e.g. tralokinumab), 50 mM sodium acetate buffer, 85 mM sodium chloride, 0.01% (w/v) polysorbate 80, wherein the pharmaceutical composition has a pH of 5.5.

### Other Definitions

Unless defined otherwise, all technical and scientific terms used herein have the same meaning as commonly understood to one of ordinary skill in the art to which this invention belongs.

The articles “a” and “an” refer to one or to more than one (i.e., to at least one) of the grammatical object of the article. By way of example, “an element” means one element or more than one element.

The terms “comprise” and “comprising” are used in the inclusive, open sense, meaning that additional elements may be included.

In general, methods “comprising” a number of steps do not require the steps to be performed in a particular order. Where a method comprises a number of sequentially numbered or alphabetical steps (e.g. (1), (2), (3); (i), (ii), (iii); or (a), (b), (c) etc.), this implies that the steps must be performed in the prescribed order unless stated otherwise.

The term “including” is used herein to mean “including but not limited to”.

The term “about” in relation to a numerical value x is optional and means, for example, x±10%.

Generally, the terms “treat”, “treating”, “treatment”, or the like, mean to alleviate (reduce, minimise, or eliminate) symptoms, or to reduce, minimise or eliminate the causation of symptoms either on a temporary or permanent basis. All publications mentioned herein are incorporated by reference in their entirety.

### EXAMPLES

The invention is further illustrated by the following examples. It will be appreciated that the examples are for illustrative purposes only and are not intended to limit the invention as described above. Modification of detail may be made without departing from the scope of the invention.

### Example 1: Efficacy and Safety of Tralokinumab Plus Topical Corticosteroids in Patients with Severe Atopic Dermatitis and Prior History of Dupilumab Treatment: A Post Hoc Subgroup Analysis from ECZTRA 7 Trial

### Methods

ECZTRA 7 was a randomized, double-blinded, multicenter, placebo-controlled Phase 3 trial.

Key inclusion criteria for ECZTRA7:
Adult patients with AD for ≥21 year with inadequate response to topical or documented systemic medication in the past year;
Disease not adequately controlled with, or patients with contraindications to, use of oral cyclosporine A;
AD involvement of ≥10% body surface area (BSA);
And EASI≥20 and IGA≥3 at screening and at baseline;
Worst daily pruritus numeric rating scale (NRS) average score of ≥4 during the week prior to baseline.

Eligible patients were randomized 1:1 to subcutaneous tralokinumab 300 mg every 2 weeks with TCS as needed or placebo with TCS as needed for a treatment period of 26 weeks, following a 600 mg loading dose on Day 0.

For this analysis, prior history of dupilumab treatment was confirmed and further details were collected via queries before trial unblinding. Dupilumab-experienced patients are defined as those with a confirmed history of dupilumab use prior to the trial, and dupilumab-refractory patients as those who discontinued dupilumab due to lack of efficacy or adverse events per queries prior to unblinding. Cochran-Mantel-Haenszel with treatment as only strata was used for analysis.

### Results

The dupilumab-experienced (n=14) and dupilumab-naïve (n=263) cohorts had comparable baseline characteristics, except that the median (IQR) age was 51.5 (43.0, 57.0) years for the dupilumab-experienced patients and 33.0 (25.0, 45.0) years for the dupilumab-naïve patients. The median (interquartile range, IQR) EASI and percent of patients with an IGA of 4 were 35.5 (24.8, 39.6) and 57.1% among dupilumab-experienced patients and 28.7 (22.4, 39.5) and 49.0% among dupilumab-naïve patients, respectively. Among dupilumab-experienced patients, baseline and clinical characteristics were similar between the tralokinumab+TCS as needed (n=6) and placebo+TCS as needed (n=8) groups. 50% of patients in each of these two groups discontinued dupilumab due to either lack of efficacy or safety concerns. Thus, among the 6 patients who had been treated with dupilumab and randomized to receive tralokinumab, there were 2 patients where the dupilumab treatment was discontinued because of conjunctivitis and 1 patient where the dupilumab treatment failed because of lack of efficacy.

TABLE 2
Baseline Characteristics

| | | | | Dupilumab-Experienced | | | | | | | | |
|---|---|---|---|---|---|---|---|---|---|---|---|---|
| | Dupilumab-Naïve | | | | | | Tralokinumab + | | | Placebo + | | |
| | All (n = 263) | | | All (n = 14) | | | TCS (n = 6) | | | TCS (n = 8) | | |
| Variable | n | Median | IQR | n | Median | IQR | n | Median | IQR | n | Median | IQR |
| AD duration | 262 | 26.0 | 18.0, 34.0 | 14 | 34.0 | 15.0, 44.0 | 6 | 17 | 15.0, 43.0 | 8 | 34.0 | 21.0, 47.5 |
| at baseline | | | | | | | | | | | | |
| (years) | | | | | | | | | | | | |
| Age at | 263 | 33.0 | 25.0, 45.0 | 14 | 51.5 | 43.0, 57.0 | 6 | 50.0 | 43.0, 56.0 | 8 | 51.5 | 42.0, 62.5 |
| baseline | | | | | | | | | | | | |
| (years) | | | | | | | | | | | | |
| BSA at | 263 | 52.0 | 35.0, 70.0 | 14 | 56.5 | 34.0, 70.0 | 6 | 58.5 | 50.0, 72.0 | 8 | 54.5 | 33.5, 65.0 |
| baseline (%) | | | | | | | | | | | | |
| DLQI at | 257 | 16.0 | 11.0, 21.0 | 14 | 15.0 | 8.0, 18.0 | 6 | 11.0 | 7.0, 16.0 | 8 | 16.5 | 10.5, 21.5 |
| baseline | | | | | | | | | | | | |
| EASI value | 261 | 28.7 | 22.4, 39.5 | 14 | 35.5 | 24.8, 39.6 | 6 | 37.3 | 29.0, 39.6 | 8 | 32.3 | 23.5, 38.9 |
| at baseline | | | | | | | | | | | | |
| SCORAD at | 261 | 68.9 | 61.5, 78.9 | 14 | 73.6 | 61.2, 77.0 | 6 | 72.6 | 58.0, 73.8 | 8 | 76.7 | 64.7, 82.2 |
| baseline | | | | | | | | | | | | |
| Worst Daily | 259 | 7.4 | 6.6, 8.3 | 14 | 6.7 | 5.4, 8.0 | 6 | 5.9 | 5.3, 7.6 | 8 | 7.4 | 6.2, 8.9 |
| Pruritus | | | | | | | | | | | | |
| NRS at | | | | | | | | | | | | |
| baseline | | | | | | | | | | | | |
| IGA = 4 | 263 | 129 (49.0) | | 14 | 8 (57.1) | | 6 | 5 (83.3) | | 8 | 3 (37.5) | |
| at baseline- | | | | | | | | | | | | |
| n (%) | | | | | | | | | | | | |

Among dupilumab-experienced patients at Week 16, 100% (n/N, 6/6) of patients receiving tralokinumab+TCS achieved EASI-75 without the use of rescue therapy, compared to 50% ( 4/8) of those receiving placebo+TCS (difference [95% CI]: 50.0 [15.4, 84.6]). Therefore, the patient who did not respond to dupilumab responded to tralokinumab. Numerically higher proportions of dupilumab-experienced patients receiving tralokinumab+TCS achieved IGA 0/1 ( 4/6, 66.7%; placebo+TCS: ⅜, 37.5%; difference: 29.2 [−21.3, 79.6]) and improvement in worst daily pruritus NRS (weekly average) ≥4 points ( 3/6, 50%; placebo+TCS: ⅜, 37.5%; difference: 12.5 [−39.7, 64.7]) at Week 16. Similarly, at Week 26, numerically higher proportions of dupilumab-experienced patients receiving tralokinumab+TCS achieved EASI-75 ( 6/6, 100%; placebo+TCS: ⅜, 37.5%; difference: 62.5 [29.0, 96.0]), IGA 0/1 ( 4/6, 66.7%; placebo+TCS: 2/8, 25%; difference: 41.7 [−6.5, 89.9]), and improvement in worst daily pruritus NRS (weekly average) ≥4 points ( 3/6, 50%; placebo+TCS: ⅜, 37.5%; difference: 12.5 [−39.7, 64.7]), compared to placebo+TCS.

TABLE 3
Binary Efficacy Endpoints - Dupilumab Experienced Subjects†

| | | Tralokinumab + | | Placebo + | | |
|---|---|---|---|---|---|---|
| Visit | Endpoint | TCS | | TCS | Difference (95 % CI) | |
| Week | EASI75 | 6/6 | (100.0%) | 4/8 (50.0%) | 50.0 | (15.4, 84.6) |
| 16 | IGA 0/1 | 4/6 | (66.7%) | 3/8 (37.5%) | 29.2 | (−21.3, 79.6) |
| | Itch | 3/6 | (50.0%) | 3/8 (37.5%) | 12.5 | (−39.7, 64.7) |
| | NRS ≥4 | | | | | |
| Week | EASI75 | 6/6 | (100.0%) | 3/8 (37.5%) | 62.5 | (29.0, 96.0) |
| 26 | IGA 0/1 | 4/6 | (66.7%) | 2/8 (25.0%) | 41.7 | (−6.5, 89.9) |
| | Itch | 3/6 | (50.0%) | 3/8 (37.5%) | 12.5 | (−39.7, 64.7) |
| | NRS ≥4* | | | | | |

†Cochran-Mantel-Haenszel analysis with treatment as only strata
*Improvement in worst daily pruritus NRS (weekly average) ≥4 points from Baseline

Through the 26 weeks, 66.7% ( 4/6) of dupilumab-experienced patients receiving tralokinumab+TCS reported any adverse event, compared to 87.5% (⅞) of those receiving placebo+TCS. One placebo patient reported 2 events of conjunctivitis, 1 mild and 1 of moderate severity; 1 tralokinumab patient reported 1 mild event of conjunctivitis. No serious adverse events occurred in either treatment group.

From a safety perspective, there were 2 patients who had previously discontinued dupilumab due to conjunctivitis; adverse events of conjunctivitis were not reported for either patient during 26 weeks of tralokinumab+TCS treatment.

TABLE 4
Adverse Events- Dupilumab Experienced Subjects - Through 26 Weeks

| | Tralokinumab + | |
|---|---|---|
| | TCS* | Placebo + TCS* |
| Any adverse event | 4/6 (66.7) | 7/8 (87.5) |
| Any serious adverse event | 0/6 (0.0) | 0/8 (0.0) |
| Conjunctivitis* | 1/6 (16.7) | 1/8 (12.5) |

### Conclusions

This post hoc subgroup analysis indicates that dupilumab-experienced patients can benefit from tralokinumab+TCS as needed. Overall frequencies of adverse events in dupilumab-experienced patients treated with tralokinumab+TCS as needed were consistent with the pooled analysis of tralokinumab Phase 2 and 3 trials.

### List of Abbreviations

AD, atopic dermatitis
AE, adverse event
AESI, adverse event of special interest
BSA, body surface area involvement
CI, confidence interval
DLQI, Dermatology Life Quality Index
EASI, Eczema Area and Severity Index
EASI-50, at least 50% reduction in Eczema Area and Severity Index score
EASI-75, at least 75% reduction in Eczema Area and Severity Index score
EQ-5D-5L, EuroQoL 5-Dimension Health Questionnaire 5 Level
HADS, Hospital Anxiety and Depression Scale
HRQoL, Health-related quality of life
IGA, Investigator's Global Assessment
IGA-0/1, Investigators' Global Assessment score of 0 (clear) or 1 (almost clear)
IMP, investigational medicinal product
IQR, interquartile range
NRS, Numeric Rating Scale
PT, preferred term
PYE, patient-years of exposure
Q2W, every other week, i.e. every 2 weeks
Q4W, every 4 weeks
R, rate (number of AEs divided by PYE multiplied by 100)
SAEs, serious adverse events
SCORAD, SCORing Atopic Dermatitis
SE, standard error
SF-36, Short Form (36) Health Survey
TCS, topical corticosteroids.

The following numbered clauses, describing aspects of the invention, are part of the description.

1. An interleukin-13 (IL-13) binding protein for use in a method of treating atopic dermatitis (AD) in a subject, wherein the AD is inadequately controlled by an agent that inhibits IL-13 and IL-4 signalling, such as an anti-IL4Rα antibody (e.g. dupilumab) and wherein the method comprises administering the IL-13 binding protein to the subject.

2. A method for treating treating atopic dermatitis (AD) in a subject in need thereof, wherein the AD is inadequately controlled by an agent that inhibits IL-13 and IL-4 signalling, such as an anti-IL4Rα antibody (e.g. dupilumab), wherein the method comprises the step of administering the IL-13 binding protein to the subject.

3. The IL-13 binding protein for use according to clause 1 or the method according to clause 2, wherein the method comprises the step of selecting a subject in which AD is inadequately controlled by an agent that inhibits IL-13 and IL-4 signalling, such as an anti-IL4Rα antibody (e.g. dupilumab).

4. An IL-13 binding protein for use in a method of treating atopic dermatitis in a subject who has experienced conjunctivitis when treated with an antibody that inhibits IL-4 and IL-13 signalling, such as an anti-IL4Rα antibody (e.g. dupilumab), wherein the method comprises administering the IL-13 binding protein to the subject.

5. A method for treating atopic dermatitis in a subject who has experienced conjunctivitis when treated with an antibody that inhibits IL-4 and IL-13 signalling, such as an anti-IL4Rα antibody (e.g. dupilumab), wherein the method comprises administering the IL-13 binding protein to the subject.

6. The IL-13 binding protein for use according to clause 4 or the method according to clause 5, wherein the method comprises the step of selecting a subject who has experienced conjunctivitis when treated with an antibody that inhibits IL-4 and IL-13 signalling, such as an anti-IL4Rα antibody (e.g. dupilumab).

7. The IL-13 binding protein for use or the method according to any one of the preceding clauses, wherein the agent that inhibits IL-13 and IL-14 signalling (e.g. dupilumab) does not result in ≥75% improvement of Eczema Area and Severity Index (EASI-75) compared to baseline.

8. The IL-13 binding protein for use or the method according to any one of the preceding clauses, wherein the AD is also inadequately controlled by cyclosporine A.

9. The IL-13 binding protein for use or the method according to any one of the preceding clauses, wherein the method comprises the steps of: (a) administering a first dose of the IL-13 binding protein to the subject; and (b) administering one or more secondary dose(s) of the IL-13 binding protein to the subject, wherein each secondary dose is administered to the subject from 12 days to 35 days after the immediately preceding dose.

10. The IL-13 binding protein for use or the method according to clause 9, wherein each secondary dose is administered to the subject about 2 weeks or about 4 weeks after the immediately preceding dose.

11. The IL-13 binding protein for use or the method according to any one of the preceding clauses, wherein the method is carried out for at least 2 weeks, at least 3 weeks, at least 12 weeks, at least 3 months, at least 16 weeks, at least 24 weeks, at least 26 weeks, at least 6 months, at least 32 weeks, at least 36 weeks, at least a year, or at least 52 weeks or more.

12. The IL-13 binding protein for use or the method according to clause 11, wherein the method is carried out for at least 26 weeks.

13. The IL-13 binding protein for use or the method according to any one of the preceding clauses, wherein the method comprises the steps of: (a) administering a first dose of about 10 to about 600 mg of the IL 13 binding protein to the subject; and (b) administering one or more secondary dose(s) of about 10 to about 600 mg of the IL-13 binding protein to the subject, wherein each secondary dose is administered to the subject from 12 days to 35 days after the immediately preceding dose.

14. The IL-13 binding protein for use or the method according to clause 13, wherein the method comprises the steps of: (a) administering a first dose of about 600 mg of the IL 13 binding protein to the subject; and (b) administering one or more secondary dose(s) of about 300 mg of the IL-13 binding protein to the subject, wherein each secondary dose is administered to the subject from 12 days to 35 days after the immediately preceding dose.

15. The IL-13 binding protein for use or the method according to clause 14, wherein the method, wherein each secondary dose is administered to the subject about 2 weeks or about 4 weeks after the immediately preceding dose.

16. The IL-13 binding protein for use or the method according to any one of the preceding clauses, wherein the AD is moderate-to-severe or severe AD.

17. The IL-13 binding protein for use or the method according to any one of the preceding clauses, wherein the IL-13 binding protein is administered subcutaneously.

18. The IL-13 binding protein for use or the method according to any one of the preceding clauses, wherein the IL-13 binding protein is administered as a pharmaceutical composition comprising 50 mM sodium acetate buffer, 85 mM sodium chloride, 0.01% (w/v) polysorbate 80, wherein the pharmaceutical composition has a pH of 5.5.

19. The IL-13 binding protein for use or the method according to any one of the preceding clauses, wherein the IL-13 binding protein is an anti-IL-13 antibody, or an IL-13-binding fragment thereof.

20. The IL-13 binding protein for use or the method according to any one of the preceding clauses, wherein the IL-13 binding protein is a monoclonal anti-IL-13 antibody, or an IL-13-binding fragment thereof.

21. The IL-13 binding protein for use or the method according to any one of the preceding clauses, wherein the IL-13 binding protein is a human anti-IL-13 antibody, or an IL-13-binding fragment thereof.

22. The IL-13 binding protein for use or the method according to any one of clauses 19-21, wherein the IL-13 antibody is an IgG4 antibody.

23. The IL-13 binding protein for use or the method according to any one of clauses 19-22, wherein the IL-13-binding fragment is selected from a Fab, Fab′, F(ab′)2, Fd, Fv, single-chain Fv (scFv), or disulfide-linked Fvs (sdFv).

24. The IL-13 binding protein for use or the method according to any one of clauses 19-23, wherein the anti-IL-13 antibody, or the IL-13-binding fragment thereof, comprises:
a heavy chain complementarity determining region 1 (HCDR1) comprising an amino acid sequence of SEQ ID NO:1;
a heavy chain complementarity determining region 2 (HCDR2) comprising an amino acid sequence of SEQ ID NO:2;
a heavy chain complementarity determining region 3 (HCDR3) comprising an amino acid sequence of SEQ ID NO:3;
a light chain complementarity determining region 1 (LCDR1) comprising an amino acid sequence of SEQ ID NO:4;
a light chain complementarity determining region 2 (LCDR2) comprising an amino acid sequence of SEQ ID NO:5; and
a light chain complementarity determining region 3 (LCDR3) comprising an amino acid sequence of SEQ ID NO:6.

25. The IL-13 binding protein for use or the method according to any one of clauses 19-24, wherein the anti-IL-13 antibody, or the IL-13-binding fragment thereof, comprises a heavy chain variable region (HCVR) and a light chain variable region (LCVR), wherein:
(i) the heavy chain variable region comprises:
a heavy chain complementarity determining region 1 (HCDR1) comprising an amino acid sequence of SEQ ID NO:1;
a heavy chain complementarity determining region 2 (HCDR2) comprising an amino acid sequence of SEQ ID NO:2; and
a heavy chain complementarity determining region 3 (HCDR3) comprising an amino acid sequence of SEQ ID NO:3; and
(ii) the light chain variable region comprises:
a light chain complementarity determining region 1 (LCDR1) comprising an amino acid sequence of SEQ ID NO:4;
a light chain complementarity determining region 2 (LCDR2) comprising an amino acid sequence of SEQ ID NO:5; and
a light chain complementarity determining region 3 (LCDR3) comprising an amino acid sequence of SEQ ID NO:6.

26. The IL-13 binding protein for use or the method according to any one of clauses 19-25, wherein the anti-IL-13 antibody, or the IL 13-binding fragment thereof, further comprises:
(i) an amino acid sequence that is at least 80%, at least 85%, at least 90%, at least 95%, at least 96%, at least 97%, at least 98%, or at least 99% identical to a heavy chain variable region sequence of SEQ ID NO:8; and/or
(ii) an amino acid sequence that is at least 80%, at least 85%, at least 90%, at least 95%, at least 96%, at least 97%, at least 98%, or at least 99% identical to a light chain variable region sequence of SEQ ID NO:10.

27. The IL-13 binding protein for use or the method according to any one of clauses 19-26, wherein the anti-IL-13 antibody, or the IL-13-binding fragment thereof, comprises a heavy chain variable region sequence of SEQ ID NO:8 and a light chain variable region sequence of SEQ ID NO:10.

28. The IL-13 binding protein for use or the method according to any one of clauses 19-27, wherein the anti-IL-13 antibody, or the IL-13-binding fragment thereof, comprises: (i) an amino acid sequence that is at least 80%, at least 85%, at least 90%, at least 95%, at least 96%, at least 97%, at least 98%, or at least 99% identical to the heavy chain sequence of SEQ ID NO:11; and/or (ii) an amino acid sequence that is at least 80%, at least 85%, at least 90%, at least 95%, at least 96%, at least 97%, at least 98%, or at least 99% identical to the light chain sequence of SEQ ID NO:12.

29. The IL-13 binding protein for use or the method according to any one of clauses 19-28, wherein the anti-IL-13 antibody, or the IL-13-binding fragment thereof, comprises a heavy chain of SEQ ID NO:11 and a light chain sequence of SEQ ID NO:12.

30. The IL-13 binding protein for use or the method according to clause 29, wherein the anti-IL-13 antibody is tralokinumab.

31. The IL-13 binding protein for use or the method according to any one of the preceding clauses, wherein the IL-13 binding protein is administered as a monotherapy.

32. The IL-13 binding protein for use or the method according to any one of the preceding clauses, wherein the IL-13 binding protein is administered in combination with a second therapeutic agent selected from the group consisting of a topical corticosteroid, a topical calcineurin inhibitor, an anti-histamine, an emollient, or an anti-bacterial therapeutic.

33. The IL-13 binding protein for use or the method according to clause 32, wherein the IL-13 binding protein is administered in combination with a topical corticosteroid.

34. The IL-13 binding protein for use or the method according to any one of the preceding clauses wherein the agent that inhibits IL-13 and IL-4 signalling is an antibody or antigen binding fragment thereof.

35. The IL-13 binding protein for use or the method according to clause 34, wherein the agent that inhibits IL-13 and IL-4 signalling is an antigen binding fragment, wherein the antigen binding fragment is selected from a Fab, Fab′, F(ab′)2, Fd, Fv, single-chain Fv (scFv), or disulfide-linked Fvs (sdFv).

36. The IL-13 binding protein for use or the method according to any one of clauses 34 or 35, wherein the agent that inhibits IL-13 and IL-4 signalling is an antibody or antigen binding fragment thereof that binds to IL-4Rα.

37. The IL-13 binding protein for use or the method according to any one of clauses 34-36, wherein the agent that inhibits IL-13 and IL-4 signalling is dupilumab.

38. The IL-13 binding protein for use or the method according to any one of the preceding clauses, wherein the method achieves ≥50% improvement of Eczema Area and Severity Index (EASI-50) compared to baseline.

39. The IL-13 binding protein for use or the method according to any one of the preceding clauses, wherein the method achieves ≥75% improvement of Eczema Area and Severity Index (EASI-75) compared to baseline.

40. The IL-13 binding protein for use or the method according to clause 39, wherein the method achieves ≥75% improvement of Eczema Area and Severity Index (EASI-75) compared to baseline at week 16.

41. The IL-13 binding protein for use or the method according to clause 39, wherein the method achieves ≥75% improvement of Eczema Area and Severity Index (EASI-75) compared to baseline at week 26.

42. The IL-13 binding for use or the method according to any one of the preceding clauses, wherein the method comprises (a) administering a first dose of about 600 mg of the IL-13 binding protein to the subject; and (b) administering one or more secondary dose(s) of about 300 mg of the IL-13 binding protein to the subject, wherein each secondary dose is administered to the subject 2 weeks or 4 weeks after the immediately preceding dose, wherein the IL-13 binding protein is an antibody comprising a heavy chain variable region sequence shown as SEQ ID NO:8 and a light chain variable region sequence shown as SEQ ID NO:10, wherein the method is carried out for at least 26 weeks and wherein the method achieves ≥75% improvement of Eczema Area and Severity Index (EASI-75) compared to baseline at week 26.

1. (canceled)

2. A method for treating treating moderate-to-severe or severe atopic dermatitis (AD) in a subject in need thereof, wherein the AD is inadequately controlled by an agent that inhibits IL-13 and IL-4 signalling, such as an anti-IL4Rα antibody (e.g. dupilumab), or wherein the subject who has experienced conjunctivitis when treated with an antibody that inhibits IL-4 and IL-13 signalling, such as an anti-IL4Rα antibody (e.g. dupilumab), wherein the method comprises the step of administering the IL-13 binding protein to the subject subcutaneously, wherein the IL-13 binding protein is tralokinumab and wherein the treatment comprises the steps of: (a) administering a first dose of about 600 mg of the IL-13 binding protein to the subject; and (b) administering one or more secondary dose(s) of about 300 mg of the IL-13 binding protein to the subject, wherein each secondary dose is administered to the subject about 2 weeks or about 4 weeks after the immediately preceding dose, and wherein the treatment is carried out for at least 2 weeks.

3. The method according to claim 1, wherein the method comprises the step of selecting a subject in which AD is inadequately controlled by an agent that inhibits IL-13 and IL-4 signalling, such as an anti-IL4Rα antibody (e.g. dupilumab).

4. (canceled)

5. (canceled)

6. The method according to claim 1, wherein the method comprises the step of selecting a subject who has experienced conjunctivitis when treated with an antibody that inhibits IL-4 and IL-13 signalling, such as an anti-IL4Rα antibody (e.g. dupilumab).

7. The method according to claim 1, wherein the agent that inhibits IL-13 and IL-14 signalling (e.g. dupilumab) does not result in ≥75% improvement of Eczema Area and Severity Index (EASI-75) compared to baseline.

8. The method according to claim 1, wherein the AD is also inadequately controlled by cyclosporine A.

9. (canceled)

10. (canceled)

11. The method according to claim 1, wherein the method is carried out for at least 2 weeks, at least 3 weeks, at least 12 weeks, at least 3 months, at least 16 weeks, at least 24 weeks, at least 26 weeks, at least 6 months, at least 32 weeks, at least 36 weeks, at least a year, or at least 52 weeks or more.

12. (canceled)

13. (canceled)

14. (canceled)

15. (canceled)

16. (canceled)

17. (canceled)

18. The method according to claim 1, wherein the tralokinumab is administered as a pharmaceutical composition comprising 50 mM sodium acetate buffer, 85 mM sodium chloride, 0.01% (w/v) polysorbate 80, wherein the pharmaceutical composition has a pH of 5.5.

19-27. (canceled)

28. The method according to claim 1, wherein the IL-13 binding protein is administered as a monotherapy.

29. The method according to claim 1, wherein the IL-13 binding protein is administered in combination with a second therapeutic agent selected from the group consisting of a topical corticosteroid, a topical calcineurin inhibitor, an anti-histamine, an emollient, or an anti-bacterial therapeutic.

30. The method according to claim 29, wherein the IL-13 binding protein is administered in combination with a topical corticosteroid.

31. (canceled)

32. (canceled)

33. The method according to claim 1, wherein the agent that inhibits IL-13 and IL-4 signalling is an antibody or antigen binding fragment thereof that binds to IL-4Rα.

34. The method according to claim 1, wherein the agent that inhibits IL-13 and IL-4 signalling is dupilumab.

35. The method according to claim 1, wherein the method achieves ≥50% improvement of Eczema Area and Severity Index (EASI-50) compared to baseline.

36. The method according to claim 1, wherein the method achieves ≥75% improvement of Eczema Area and Severity Index (EASI-75) compared to baseline.

37. The method according to claim 36, wherein the method achieves ≥75% improvement of Eczema Area and Severity Index (EASI-75) compared to baseline at week 16.

38. The method according to claim 36, wherein the method achieves ≥75% improvement of Eczema Area and Severity Index (EASI-75) compared to baseline at week 26.

39-41. (canceled)
